(19) United States
(12) Patent Application Publication
(10) Pub. No.: US 2025/0312040 A1
(43) Pub. Date: Oct. 9, 2025

(54) SENSOR ARRANGEMENTS FOR TISSUE PRESENCE AND PLACEMENT DETECTION IN A SURGICAL INSTRUMENT

(71) Applicant: Cilag GmbH International, Zug (CH)

(72) Inventor: Mark Overmyer, Cincinnati, OH (US)

(73) Assignee: Cilag GmbH International, Zug (CH)

(21) Appl. No.: 18/627,608

(22) Filed: Apr. 5, 2024

(51) Int. Cl. A61B 17/072; A61B 17/00

(52) U.S. Cl. CPC A61B 17/07207; A61B 2017/00026; A61B 2017/07271; A61B 2017/07285

(57) ABSTRACT

An end effector of a surgical instrument may be equipped with a limited (e.g., minimal) number of electrical pads that provides useful tissue presence and positioning information using a limited number of signals communicated between the electrical pads and a controller located remotely from the end effector.

### BACKGROUND

In some settings, laparoscopic or endoscopic surgical instruments may be preferred over traditional open surgical instruments to minimize the size of the surgical incision as well as post-operative recovery time and complications. Consequently, some endoscopic surgical instruments may be suitable for placement of a distal end effector at a desired surgical site through the cannula of a trocar. These distal end effectors may engage tissue in a number of ways to achieve a diagnostic or therapeutic effect (e.g., endocutter, grasper, cutter, stapler, clip applier, access device, drug/gene therapy delivery device, and energy delivery device using ultrasound, RF, laser, etc.). Endoscopic surgical instruments may include a shaft that extends proximally from the end effector to a handle portion, which is manipulated by the clinician, or alternatively to a robot. Such a shaft may enable insertion to a desired depth and rotation about the longitudinal axis of the shaft, thereby facilitating positioning of the end effector within the patient. Positioning of an end effector may be further facilitated through inclusion of one or more articulation joints or features, enabling the end effector to be selectively articulated or otherwise deflected relative to the longitudinal axis of the shaft.

Examples of endoscopic surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Such endoscopic surgical staplers may also be used in open procedures and/or other non-endoscopic procedures. By way of example only, a surgical stapler may be inserted through a thoracotomy and thereby between a patient's ribs to reach one or more organs in a thoracic surgical procedure that does not use a trocar as a conduit for the stapler. Such procedures may include the use of the stapler to sever and close a vessel leading to an organ, such as a lung. For instance, the vessels leading to an organ may be severed and closed by a stapler before removal of the organ from the thoracic cavity. Of course, surgical staplers may be used in various other settings and procedures.

In some such procedures, the clinician may have a need or desire for feedback regarding the presence and position of clamped tissue within the end effector during the surgical procedure to better understand whether the tissue is properly clamped.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a perspective view of an example of an articulating surgical stapling instrument;

FIG. 2 depicts a side view of the instrument of FIG. 1;

FIG. 3 depicts a perspective view of an opened end effector of the instrument of FIG. 1;

FIG. 4A depicts a side cross-sectional view of the end effector of FIG. 3, taken along line 4-4 of FIG. 3, with the firing beam in a proximal position;

FIG. 4B depicts a side cross-sectional view of the end effector of FIG. 3, taken along line 4-4 of FIG. 3, with the firing beam in a distal position;

FIG. 5 depicts an end cross-sectional view of the end effector of FIG. 3, taken along line 5-5 of FIG. 3;

FIG. 6 depicts an exploded perspective view of the end effector of FIG. 3;

FIG. 7 depicts a perspective view of the end effector of FIG. 3, positioned at tissue and having been actuated once in the tissue;

FIG. 8 depicts a perspective view of an alternative version of an end effector with an angled anvil jaw and an angled cartridge;

FIG. 9 depicts a perspective view of an end effector equipped with a sensor circuit that may be used with a surgical instrument such as the surgical instrument of FIG. 1;

FIG. 10 is a diagram depicting an example sensor circuit that may be used with an end effector such as the end effector of FIG. 9;

FIG. 11A is a diagram depicting another example sensor circuit that may be used with an end effector such as the end effector of FIG. 9;

FIG. 11B is a diagram depicting yet another example sensor circuit that may be used with an end effector such as the end effector of FIG. 9;

FIG. 12 is a diagram depicting still another example sensor circuit that may be used with an end effector such as the end effector of FIG. 9; and

FIG. 13 is a flowchart of an example method for determining positioning of tissue grasped between a first jaw and a second jaw of an end effector of a surgical instrument.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those having ordinary skill in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

For clarity of disclosure, the terms “proximal” and “distal” are defined herein relative to a human or robotic operator of the surgical instrument. The term “proximal” refers to the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “lateral,” “transverse,” “bottom,” “top,” are relative terms to provide additional clarity to the figure descriptions provided below. The terms “upper,” “lower,” “lateral,” “transverse,” “bottom,” “top,” are thus not intended to unnecessarily limit the invention described herein.

Furthermore, the terms “about,” “approximately,” “substantially,” and the like as used herein in connection with any numerical values, ranges of values, and/or geometric/positional quantifications are intended to encompass the exact value(s) or quantification(s) referenced as well as a suitable tolerance that enables the referenced feature or combination of features to function for the intended purpose described herein. For example, “substantially parallel” encompasses nominally parallel structures.

As used herein in connection with various examples of end effector jaw tips, a tip described as “angled,” “bent,” or “curved” encompasses tip configurations in which a longitudinal path (e.g., linear or arcuate) along which the tip extends is non-coaxial and non-parallel with a longitudinal axis of the jaw body; particularly, configurations in which the longitudinal tip path extends distally toward the opposing jaw. Conversely, a tip described as “straight” encompasses tip configurations in which a longitudinal axis of the tip is substantially parallel or coaxial with the longitudinal axis of the jaw body.

### I. Illustrative Surgical Stapler

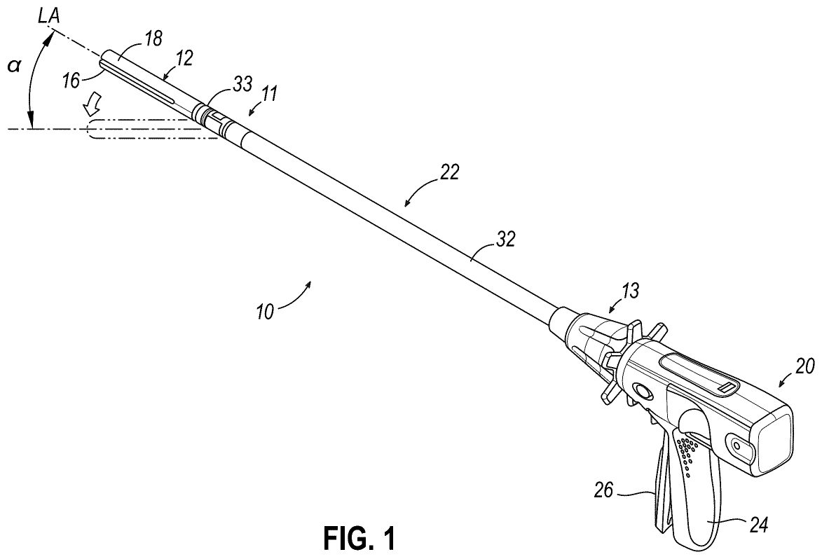

FIGS. 1-7 depict an example of a surgical stapling and severing instrument 10 that is sized for insertion through a trocar cannula or an incision (e.g., thoracotomy, etc.) to a surgical site in a patient for performing a surgical procedure. Instrument 10 of the present example includes a handle portion 20 connected to a shaft 22, which distally terminates in an articulation joint 11, which is further coupled with an end effector 12. Once articulation joint 11 and end effector 12 are inserted through the cannula passageway of a trocar, articulation joint 11 may be remotely articulated, as depicted in phantom in FIG. 1, by an articulation control 13, such that end effector 12 may be deflected from the longitudinal axis (LA) of shaft 22 at a desired angle (α). End effector 12 of the present example includes a lower jaw 16 (also referred to herein as a cartridge jaw) that includes a staple cartridge 37, and an upper jaw 18 in the form of a pivotable anvil jaw.

Unless otherwise described, the term “pivot” (and variations thereof) as used herein encompasses but is not necessarily limited to pivotal movement about a fixed axis. For instance, in some versions, upper jaw 18 may pivot about an axis that is defined by a pin (or similar feature) that slidably translates along an elongate slot or channel as upper jaw 18 moves toward lower jaw 16. Such translation may occur before, during, or after the pivotal motion. It should therefore be understood that such combinations of pivotal and translational movement are encompassed by the term “pivot” and variations thereof as used herein.

Handle portion 20 includes a pistol grip 24 and a closure trigger 26. Closure trigger 26 is pivotable toward pistol grip 24 to cause clamping, or closing, of upper jaw 18 toward lower jaw 16 of end effector 12. Such closing of upper jaw 18 is provided through a closure tube 32 and a closure ring 33, which both longitudinally translate relative to handle portion 20 in response to pivoting of closure trigger 26 relative to pistol grip 24. Closure tube 32 extends along the length of shaft 22; and closure ring 33 is positioned distal to articulation joint 11. Articulation joint 11 is operable to communicate/transmit longitudinal movement from closure tube 32 to closure ring 33.

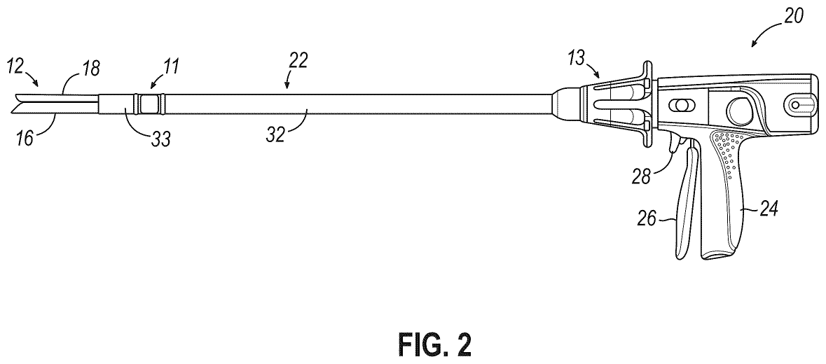

As shown in FIG. 2, handle portion 20 also includes a firing trigger 28. An elongate member (not shown) longitudinally extends through shaft 22 and communicates a longitudinal firing motion from handle portion 20 to a firing beam 14 in response to actuation of firing trigger 28. This distal translation of firing beam 14 causes the stapling and severing of clamped tissue in end effector 12, as will be described in greater detail below.

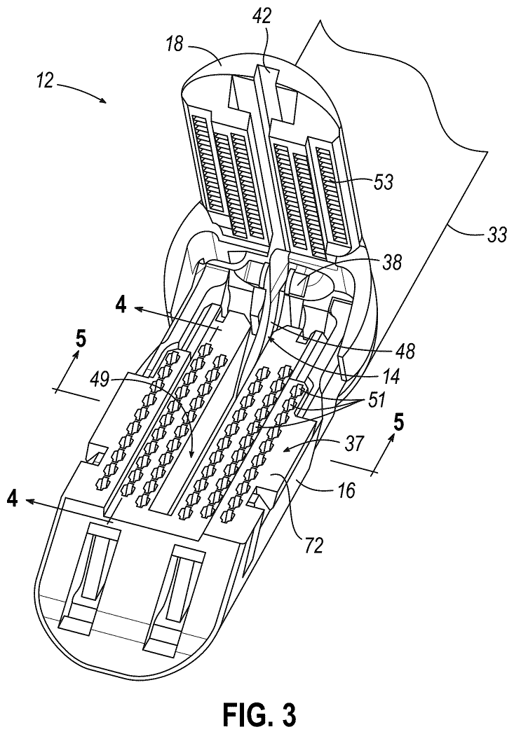

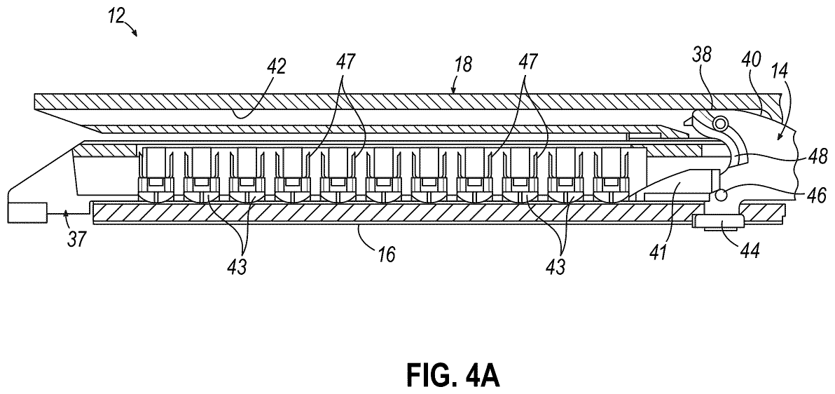

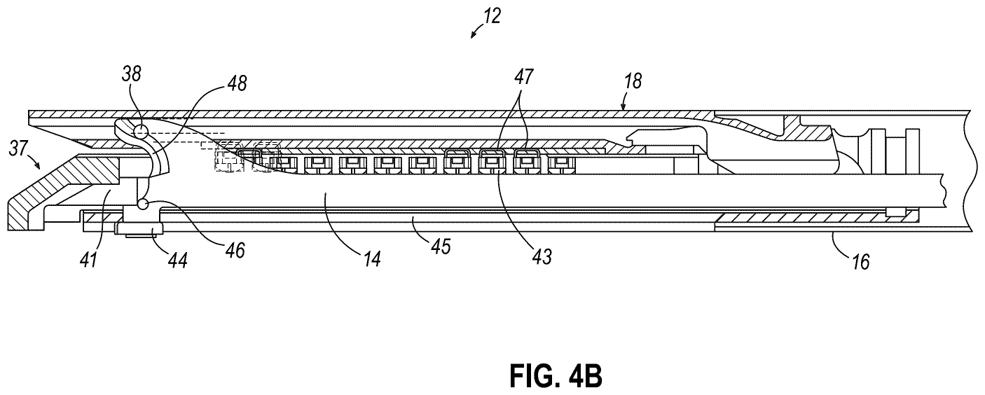

As shown in FIGS. 3-6, end effector 12 employs a firing beam 14 that includes a transversely oriented upper pin 38, a firing beam cap 44, a transversely oriented middle pin 46, and a distally presented cutting edge 48. Upper pin 38 is positioned and translatable within a longitudinal anvil slot 42 of upper jaw 18. Firing beam cap 44 slidably engages a lower surface of lower jaw 16 by having firing beam 14 extend through lower jaw slot 45 (shown in FIG. 4B) that is formed through lower jaw 16. Middle pin 46 slidingly engages a top surface of lower jaw 16, cooperating with firing beam cap 44.

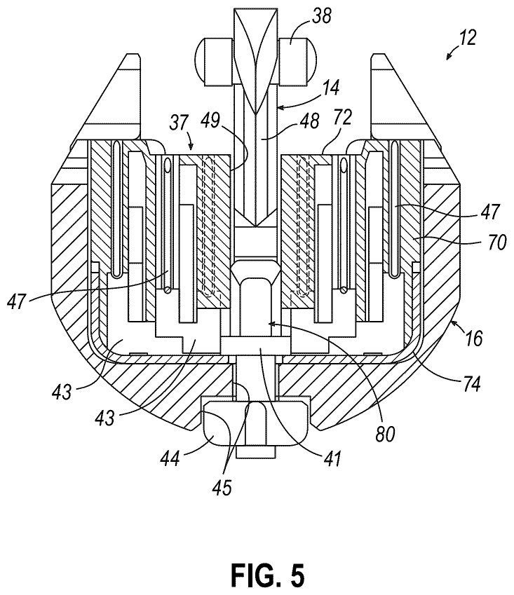

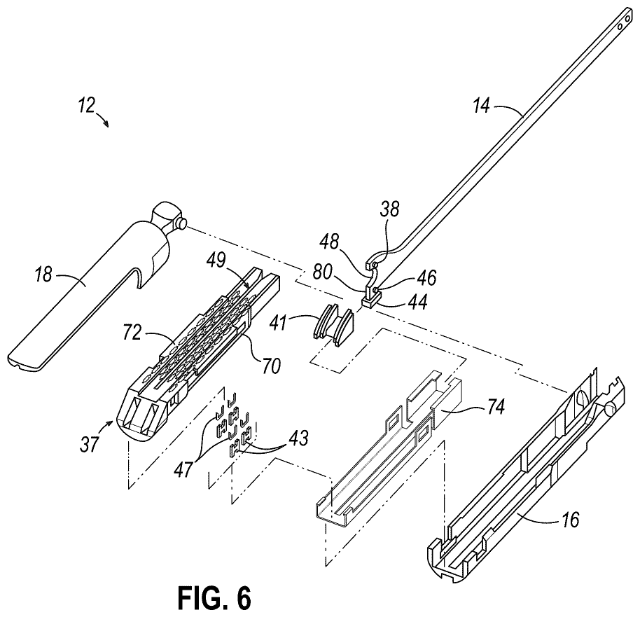

FIG. 3 shows firing beam 14 of the present example proximally positioned and upper jaw 18 pivoted to an open configuration, allowing an unspent staple cartridge 37 to be removably installed into a channel of lower jaw 16. As best seen in FIGS. 5-6, staple cartridge 37 of the present example includes a cartridge body 70, which presents an upper deck 72 and is coupled with a lower cartridge tray 74. As best seen in FIG. 3, a vertical slot 49 extends longitudinally through a portion of staple cartridge body 70. As also best seen in FIG. 3, three rows of staple apertures 51 are formed through upper deck 72 on each lateral side of vertical slot 49. As shown in FIGS. 4A-6, a wedge sled 41 and a plurality of staple drivers 43 are captured between cartridge body 70 and tray 74, with wedge sled 41 being located proximal to staple drivers 43. Wedge sled 41 is movable longitudinally within staple cartridge 37; while staple drivers 43 are movable vertically within staple cartridge 37. Staples 47 are also positioned within cartridge body 70, above corresponding staple drivers 43. Each staple 47 is driven vertically within cartridge body 70 by a staple driver 43 to drive staple 47 out through an associated staple aperture 51. As best seen in FIGS. 4A-4B and 6, wedge sled 41 presents inclined cam surfaces that urge staple drivers 43 upwardly as wedge sled 41 is driven distally through staple cartridge 37.

With end effector 12 closed, as depicted in FIGS. 4A-4B by distally advancing closure tube 32 and closure ring 33, a firing member in the form of firing beam 14 is then advanced distally into engagement with upper jaw 18 by having upper pin 38 enter longitudinal anvil slot 42. A pusher block 80 (shown in FIG. 5) located at distal end of firing beam 14 pushes wedge sled 41 distally as firing beam 14 is advanced distally through staple cartridge 37 when firing trigger 28 is actuated. During such firing, cutting edge 48 of firing beam 14 enters vertical slot 49 of staple cartridge 37, severing tissue clamped between staple cartridge 37 and upper jaw 18. As shown in FIGS. 4A-4B, middle pin 46 and pusher block 80 together actuate staple cartridge 37 by entering into vertical slot 49 within staple cartridge 37, driving wedge sled 41 into upward camming contact with staple drivers 43, which in turn drives staples 47 out through staple apertures 51 and into forming contact with staple forming pockets 53 (shown in FIG. 3) on inner surface of upper jaw 18. FIG. 4B depicts firing beam 14 fully distally translated after completing severing and stapling of tissue. Staple forming pockets 53 are intentionally omitted from the view in FIGS. 4A-4B but are shown in FIG. 3. Upper jaw 18 is intentionally omitted from the view in FIG. 5.

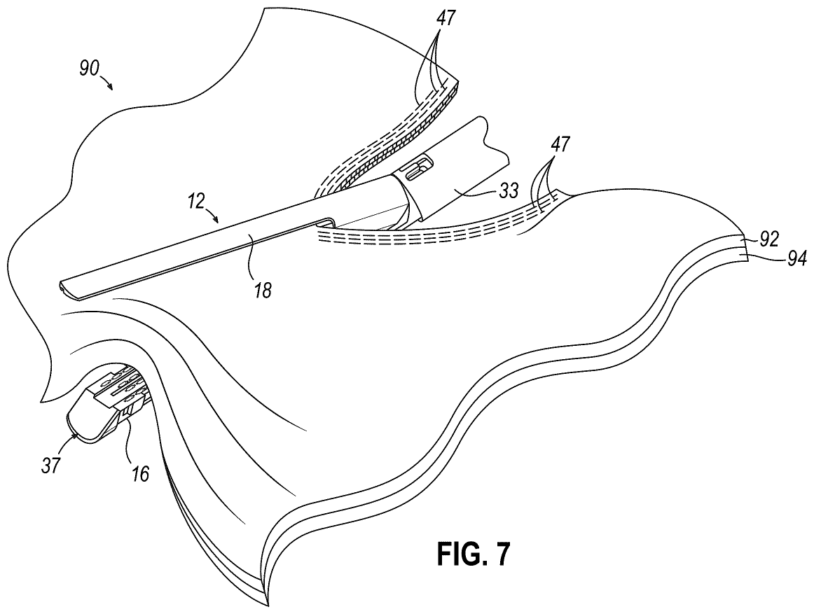

FIG. 7 shows end effector 12 having been actuated through a single firing stroke through tissue 90. Cutting edge 48 (obscured in FIG. 7) has cut through tissue 90, while staple drivers 43 have driven three alternating rows of staples 47 through tissue 90 on each side of the cut line produced by cutting edge 48. In some situations, if further cutting and stapling is desired after the first firing stroke is complete, end effector 12 is withdrawn from the patient, spent staple cartridge 37 is replaced with a new staple cartridge 37, and end effector 12 is then again inserted into the patient to reach the stapling site for further cutting and stapling. This process may be repeated until the desired quantity and pattern of firing strokes across the tissue 90 has been completed.

Instrument 10 may be further constructed and operable in accordance with any of the teachings of the following references, the disclosures of which are incorporated by reference herein: U.S. Pat. No. 8,210,411, entitled “Motor-Driven Surgical Instrument,” issued Jul. 3, 2012; U.S. Pat. No. 9,186,142, entitled “Surgical Instrument End Effector Articulation Drive with Pinion and Opposing Racks,” issued on Nov. 17, 2015; U.S. Pat. No. 9,517,065, entitled “Integrated Tissue Positioning and Jaw Alignment Features for Surgical Stapler,” issued Dec. 13, 2016; U.S. Pat. No. 9,622,746, entitled “Distal Tip Features for End Effector of Surgical Instrument,” issued Apr. 18, 2017; U.S. Pat. No. 9,717,497, entitled “Lockout Feature for Movable Cutting Member of Surgical Instrument,” issued Aug. 1, 2017; U.S. Pat. No. 9,795,379, entitled “Surgical Instrument with Multi-Diameter Shaft,” issued Oct. 24, 2017; U.S. Pat. No. 9,808,248, entitled “Installation Features for Surgical Instrument End Effector Cartridge,” issued Nov. 7, 2017; U.S. Pat. No. 9,839,421, entitled “Jaw Closure Feature for End Effector of Surgical Instrument,” issued Dec. 12, 2017; and/or U.S. Pat. No. 10,092,292, entitled “Staple Forming Features for Surgical Stapling Instrument,” issued Oct. 9, 2018.

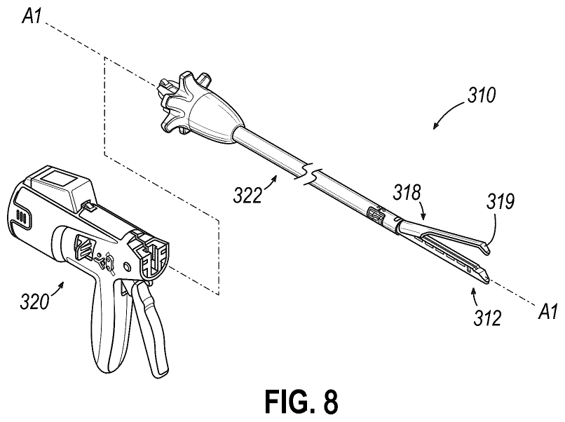

FIG. 8 shows another example of an instrument 310 configured as a surgical stapler. Instrument 310 includes a handle portion 320 and a shaft 322. Instrument 310 has a modular configuration such that shaft 322 is selectively removable from, and attachable to, handle portion 320. Instrument 310 is configured similarly to instrument 10 such that the operability and use of instrument 310 is the same as described above for instrument 10 with the added feature of instrument 310 being a modular configuration. With its modular configuration, instrument 310 provides a way to change the end effector. Such a change in the end effector may be made to replace an otherwise worn end effector, or to provide for a different end effector configuration based on the procedure or user preference. In addition to or in lieu of the foregoing, features operable for providing the modular configuration of instrument 310 may be configured in accordance with at least some of the teachings of U.S. Pat. No. 10,182,813, entitled “Surgical Stapling Instrument with Shaft Release, Powered Firing, and Powered Articulation,” issued Jan. 22, 2019, the disclosure of which is incorporated by reference herein. Other suitable components, features, and configurations for providing instrument 310 with a modular configuration will be apparent to those of ordinary skill in the art in view of the teachings herein. Moreover, it will be understood by those of ordinary skill in the art in view of the teachings herein, that instrument 10 may be modified to incorporate a modular configuration as shown and described with respect to instrument 310 or other instruments incorporated by reference herein.

In the illustrated example of FIG. 8, instrument 310 includes an end effector 312 having an upper jaw 318 that has an angled distal tip 319. It will be appreciated that end effector 312 may be used in place of end effector 12 shown in FIG. 1 or end effector 12 may be used in place of end effector 312. In some versions, end effector 312 may be integrally formed with shaft 22 or alternatively may be separately formed and then combined. In some versions, end effector 312 may be provided for use in robotic systems. In such robotic systems, modular shaft 322 having end effector 312 may be attachable to a portion of the robotic system for use such that handle portion 320 is replaced by components of the robotic system. Still in other examples, end effector 312 may be adapted for use with a robotic system in a manner where end effector 312 connects with the robotic system without necessarily connecting the entire modular shaft 322. In view of the teachings herein, other ways to incorporate an end effector having an angled elastically deformable anvil tip into a user operated or robotic operated instrument will be apparent to those of ordinary skill in the art.

### II. Sensor Arrangements for Tissue Presence and Placement Detection in a Surgical Instrument

In at least some situations, a user of a surgical instrument, such as a clinician or a robot using the surgical instrument to perform surgery or other procedure on a patient, may wish to ensure that tissue is present and fully contained within a target region (e.g., a stapling region) between jaws of an end effector of the surgical instrument, prior to firing of the end effector. Ensuring that the tissue is present and fully contained within the target region between jaws of the end effector prior to firing of the end effector may lead to more optimal cutting and stapling of the tissue when the user operates to fire the end effector. For example, in a surgical procedure in which an end effector is used to cut and seal a blood vessel, the user may wish to ensure that the blood vessel is present in the stapling region of the end effector and that the blood vessel is entirely clamped within the staple line of a cartridge of the end effector. Ensuring the blood vessel is entirely clamped within the staple line of the cartridge of the end effector may ensure that the ends of the blood vessel are properly sealed when cut and stapled by the end effector.

Generally, if the tissue is not fully contained in the target region between the jaws of the end effector, the tissue may be sub-optimally cut and/or sub-optimally stapled when the user operates to fire the end effector. If, for example, the tissue extends distally outside of the target region between the jaws of the end effector, then a single firing of the end effector may not cut the entirety of the tissue. In this case, an additional cartridge may be needed to finish cutting and stapling of the tissue, which would increase the cost of the procedure. Alternatively, a tag of the uncut tissue may remain in the body of the patient, which may lead to complications after the procedure. As another example, if the tissue extends proximally outside of the target region between the jaws of the end effector, then the tissue may be gathered or bunched up in the proximal end of the end effector, which may result in sub-optimal stapling by the end effector.

In various embodiments described below, electrical contacts or pads (or electrical contact or pad pairs) may be provided on one or more tissue engaging surfaces of the end effector of the surgical instrument. The electrical contacts (also sometimes referred to herein as “electrical pads”) may be used to detect presence and placement (also sometimes referred herein as “location” or “positioning”) of tissue in the target region between the jaws of the end effector of the surgical instrument. For example, as described in more detail below, a controller may detect tissue presence and placement based on measuring electrical characteristics (e.g., impedance, capacitance inductance, etc.) based on signals received from the electrical contact pairs as the end effector clamps down on the tissue. However, there may be limited amount of space for the electrical contacts to be placed in the end effector of the surgical instrument. Further, there may be a limited number of signals that can be communicated between electrical contacts provided in the end effector of the surgical instrument and a controller that may be located remotely from the end effector, for example in a handle of the surgical instrument.

In various embodiments, the end effector may be equipped with a limited (e.g., minimal) number of electrical contacts that provides useful tissue presence and positioning information using a limited number of signals communicated between the electrical contacts and the controller located remotely from the end effector. As just an example, only two “target” electrical contact pairs may be placed within the target region of the end effector. The target electrical contact pairs may extend (e.g., longitudinally or laterally) in, respectively, a distal half and a proximal half of the target region of the end effector. The target electrical contact pairs may provide signals indicative of whether tissue is present within the distal half and the proximal half of the target region of the end effector. Further, respective “external” electrical contact pairs may be placed externally adjacent to each of a proximal and a distal end of the target region of the end effector. Electrical contacts of the external electrical contact pair may be electrically connected with each other so that a single signal is needed to indicate whether tissue extends either distally or proximally outside of the target region of the end effector. In this way only three signals are used to detect whether tissue is present between the jaws of the end effector and whether the tissue is fully contained within the target region of the end effector. In other examples, other minimal electrical contact arrangements may be used.

Indications of whether tissue is present in the target region of the end effector and whether the tissue is fully contained with the target region of the end effector may be provided to the user. Such indications may allow the user to make appropriate adjustments, such as repositioning of the end effector, prior to firing. These and other techniques and arrangements described herein may, in at least some scenarios, use a limited number of electrical contacts and signals to provide useful tissue placement information to the user of the surgical instrument, which may lead to cost savings and/or better surgical outcomes for the patient.

It is noted that although embodiments of the present disclosure are generally described herein with reference to enabling a user to ensure that tissue (e.g., a blood vessel) to be cut is fully contained within a stapling region of an end effector of a surgical instrument, the present disclosure is not so limited. For example, the disclosed sensor arrangements may be used to provide tissue positioning information other than the tissue being fully contained within the stapling region of the end effector, in some embodiments. As an example, in a surgical procedure in which the end effector is used to cut and staple a stomach, the disclosed sensor arrangements may provide tissue positioning information that allows the user to ensure that the stomach tissue does not extend beyond the proximal end of the stapling region of the end effector. The user may thus ensure that, although stomach tissue may be extending beyond the distal end of the end effector, the stomach tissue is not gathered or bunched up in the proximal end of the end effector. In this case, although it may not be possible to cut the entirety of the stomach with a single firing of the end effector, ensuring that the stomach tissue is not gathered or bunched up in the proximal end of the end effector may lead to proper stapling of the tissue when the user operates to fire the end effector. In other examples, the disclosed sensor arrangements may be used to provide useful tissue presence and placement information in other suitable scenarios, such as procedures in which the end cutter may be used to perform procedures other than blood vessel or stomach cutting and stapling in a body of a patient.

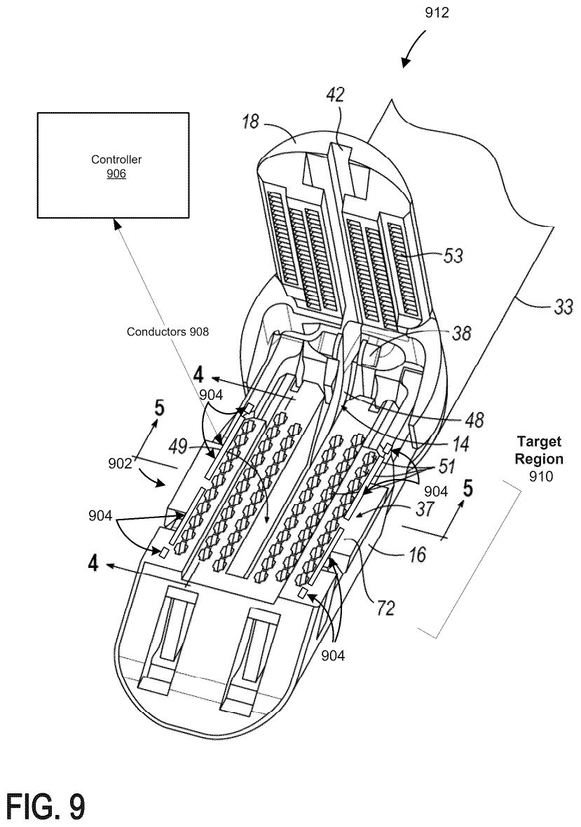

FIG. 9 depicts a perspective view of an end effector 912 that may be used with a surgical instrument such as surgical instrument 10. The end effector 912 is generally the same as the end effector 12 and includes same-numbered elements with the end effector 12 which are not discussed here again for the purpose of brevity. The end effector 912 is further equipped with a sensor circuit 902. It is noted that although the sensor circuit 902 is generally described herein in connection with the end effector 912 and the surgical instrument 10, sensor circuits the same as or similar to the sensor circuit 902 may be used with end effectors different from the end effector 912 and/or with surgical tools different from surgical instrument 10, in other examples. As just an example, the end effector 312 of the surgical tool 310 of FIG. 8 may be equipped with a sensor circuit the same as or similar to the sensor circuit 902.

The sensor circuit 902 includes a plurality of electrical contacts 904, each of the plurality of electrical contacts 904 disposed at least partially on one or more tissue contacting surfaces of the end effector 912. In some examples, the plurality of electrical contacts 904 may be disposed on an upper surface of a lower jaw 16 of the end effector 912, such as that shown in FIG. 9. For example, the electrical contacts 904 may be disposed along the cartridge body 70 of the staple cartridge 37 installed into the lower jaw 16 of the end effector 12. In other examples, at least some of the electrical contacts 904 may be disposed on a portion of the lower jaw 16 other than the cartridge body 70 of the staple cartridge 37. For example, at least some of the electrical contacts 904 may be disposed on the channel of the lower jaw 16 into which the staple cartridge 37 is installed. In this case, the electrical contacts 904 that are disposed on the channel of the lower jaw 16 into which the staple cartridge 37 is installed may be re-used with multiple staple cartridge installations into the lower jaw 16. In another example, the plurality of electrical contacts 904 may be disposed on a lower surface of the upper jaw 18 of the end effector 912. In yet another example, the plurality of electrical contacts 904 may be disposed on both the upper surface of the lower jaw 16 and the lower surface of the upper jaw 18 of the end effector 912. For example, at least some of the electrical contacts 904 illustrated in FIG. 9 may be disposed on the lower surface of the upper jaw 18.

The electrical contacts 904 may be positioned longitudinally along the length of the end effector 912 between a proximal end and a distal end of the end effector 912. In another example, other suitable placements of the electrical contacts 904 may be used. For example, suitable arrangements that are not purely longitudinal may be used. In some examples, the electrical contacts 904 may be positioned laterally in the proximal end and the distal end of the end effector 912. The electrical contacts 904 may be electrically connected (e.g., wired) to a controller 906 via conductors (e.g., wires) 908. The controller 906 may be configured to detect, using the electrical contacts 904, electrical characteristics of tissue, or other substances, in contact (e.g., conductive contact) with the electrical contacts 904 or positioned in sufficient proximity to the electrical contacts 904 to enable capacitive or indictive sensing of the tissue or the other substances. For example, the controller 906 may be configured to detect impedance, capacitance, inductance, etc. of tissue, or other substances, in contact with (or positioned sufficiently close to) the electrical contacts 904. The electrical contacts 904 may include one or more target electrical contacts 904 positioned within a target region 910 of the end effector 912, which defines tissue placement between the jaws of the end effector 912, and one or more external electrical contacts 904 positioned externally adjacent to the target region 910 of the end effector 912. The controller 906 may be configured to detect, using the one or more electrical contacts 904, presence and positioning of tissue between the jaws of the end effector 912 when the end effector 912 clamps down to grasp the tissue. For example, the controller 906 may be configured to detect, using the one or more target electrical contacts 904, whether tissue is present within the target region 910 and, in some implementations, whether the tissue is well centered within the target region 910. As another example, the controller 906 may be configured to detect, using the one or more external electrical contacts 904, whether tissue that is present within the target region 910 is entirely contained within the target region 910 or whether the tissue extends beyond the target region 910 at one or both of distal end and the proximal end of the target region 910.

The target region 910 may correspond to an optimal region within which tissue (e.g., a vessel) that a user of the surgical instrument 10 (e.g., a clinician or a robot) wishes to cut and staple should be positioned for optimal surgical outcome, for example. For example, the target region 910 may correspond to a stapling range of the end effector 912. The target region 910 may be located longitudinally in a middle portion of the end effector 912 and may cover, for example, approximately 80% (or a suitable portion of another size) of the length of the end effector 912. In an example, if tissue that the user wishes to cut and staple is contained entirely within the target region 910 and is relatively well centered in the target region 910, then the entirety of the tissue may be cut and optimally stapled in a single firing of the end effector 912. On the other hand, if the tissue that the user wishes to cut and staple is not contained entirely within the target region 910 and/or is not well centered in the target region 910, then the tissue may not be cut in its entirety in a single firing of the end effector 912 and/or may be sub-optimally stapled by the end effector 912. For example, if the tissue that the user wishes to cut and staple extends distally beyond the target region 910 of the end effector 912, then a portion of the tissue may remain uncut after a single firing of the end effector 912, which may leave a hanging tissue tag in a body of a patient after a single firing of the end effector 912. In this case, the user may need to replace the staple cartridge 37 with a new staple cartridge to finish cutting and stapling of the tissue, thereby increasing the cost of the procedure. Alternatively, the user may decide to leave the tag uncut, which may lead to suboptimal surgical outcome. As another example, if the tissue that the user wishes to cut and staple is not well centered in the target region 910 of the end effector 912 and is, for example, bunched up at the proximal end of the end effector 912, then the bunched up tissue may be sub-optimally stapled by the end effector 912.

The controller 906 may be configured to provide indications regarding the detected presence and positioning of the tissue between the jaws of the end effector 912 to the user before the user actuates to fire the end effector 912. Such information may allow the user to make appropriate decisions prior to actuation of the end effector 912. For example, the user may decide to reposition the end effector 912 to ensure that the tissue that the user wishes to cut and staple is entirely contained within the target region 910 and/or to better center the tissue that the user wishes to cut and staple within the target region 910. After the user repositions the end effector 912, indications provided by the controller 906 may inform the user that the tissue is now contained entirely within target the region 910 and/or is well centered in target region 910, assuring the user that the entirety of the tissue will be cut and optimally stapled in a single firing when the user actuates the end effector 912.

In various examples, the electrical contacts 904 comprise pads mounted on, or otherwise disposed on and/or attached to, one or more tissue contacting surfaces of end effector 912. For example, the electrical contacts 904 may be composed of a conductive material printed on the one or more tissue contacting surfaces of end effector 912. The conductive pads may enable galvanic sensing to be performed using the electrical contacts 904. In another example, the electrical contacts 904 may comprise electrical pads that are coated in a non-conductive material. The electrical pads may thus be non-conductive, galvanically isolated pads, that may enable non-galvanic (e.g., capacitive or inductive) sensing to be performed using the electrical contacts 904. It is noted that although, for ease of explanation, detection of tissue presence and placement are generally described herein with reference to the tissue being “in contact with” electrical contacts, the tissue need not necessarily be in contact with the electrical contacts. For example, in some embodiments, that tissue may be in sufficiently close proximity with the electrical pads (e.g., non-conductive pads), but not necessarily in contact with the electrical pads, such that the presence and placement of tissue may be detected based on capacitive or indictive sensing, for example.

In an example, portions of the conductive material may be covered with an insulating material, leaving the remaining uncovered portions to form the electrical contacts 904. In another example, the conductive material may be composed of metal particles bonded to the one or more tissue contacting surfaces of the end effector 912 which form an electrical circuit connecting the electrical contacts 904. The printed electrical circuit may be printed onto the one or more tissue contacting surfaces of the end effector 912 using a three-dimensional printer, for example. In various examples, the electrical contacts 904 comprise electrodes, or other electrical contacts, that are printed onto, or otherwise formed on, the one or more tissue contacting surfaces of end effector 912.

The electrical contacts 904 may include a polygonal surface configured to contact the tissue. In another example, the electrical contacts 904 may include a curved and/or tortuous shaped surface which, in various instances, may increase the contact area between the electrical contacts 904 and the tissue. In an example, the electrical contacts 904 may comprise needles extending therefrom which are configured to penetrate the tissue. The needles comprise a diameter of about 1 micrometer (μm), for example. In various instances, the needles may improve the sensitivity of the electrical contacts 904. In an example, a conductive grease or conductive viscous agent may cover the tissue contact points of the electrical contacts 904 which may improve the contact between the electrical contacts 904 and the tissue. In other examples, the sensor circuit 902 and/or the electrical contacts 904 may be formed on the one or more tissue contacting surfaces of the end effector 912 in other suitable manners.

It is noted that although the sensor circuit 902 is generally described herein as including electrical contacts 904 that are wired to the controller 906 via conductors 908, the sensor circuit 902 may include sensor devices other that electrical contacts 904 and/or connections other than wired connections may be provided between sensor devices of the sensor circuit 902 and the controller 906. For example, in some implementations, the sensor circuit 902 may include, or may be coupled to, a controller circuit, that may include a wireless transceiver, configured to wirelessly receive signals from controller 906, to distribute the received signals to electrical contacts 904, and to wirelessly transmit signals obtained from electrical contacts 904 to controller 906. As another example, in some implementations, electrical contacts 904 may be replaced with active sensor devices configured to measure characteristics of tissue indicative of tissue presence and position in the end effector 912, and to provide the measured tissue characteristics to the controller 906 via wired or wireless connections between the sensor devices and the controller 906.

The controller 906 may be configured to detect whether the electrical contacts 904 are in contact with (or in sufficient proximity to) tissue based on monitoring a tissue characteristic (e.g., impedance, capacitance, inductance, etc.) measured using the electrical contacts 904 during clamping down of the end effector 912. For example, the controller 906 may be configured to measure tissue characteristic (e.g., impedance, capacitance, inductance, etc.) using a particular electrical contact 904 that may indicate whether the particular electrical contact 904 is in contact with tissue or with a substance other than tissue (e.g., blood). In an example, the controller 906 is configured to provide excitation signals, such as alternating current (AC) signals, to respective electrical contacts 904, and to measure a response of the tissue to the stimulus as the end effector 912 clamps down to grasp tissue. In some embodiments, the controller 906 may be configured to perform a frequency sweep by changing the frequency of the AC signal, and measuring the tissue characteristic for the different frequencies. In other examples, the controller 906 may be configured to measure the tissue characteristic at only a single frequency of interest. Such frequency measurements may indicate, in addition to presence of tissue between the jaws of the end effector 912, certain characteristics of the tissue that is present between the jaws of the end effector 912, such as whether the tissue has cancerous elements or fatty deposits, for example. The controller 906 may be configured to provide such indications to a user of the surgical instrument 10 to enable the user to make further decisions based on the tissue characteristics.

The controller 906 may include any suitable analog and/or digital circuitry configured to detect tissue presence and placement (e.g., location or position) between jaws of the end effector 912 using the electrical contacts 904 and to provide indications of tissue presence and/or placement to the user of the surgical instrument 10. For example, the controller 906 may be a fully analogue controller that may include a source circuit (e.g., current or voltage source) configured to provide excitation signals to the electrical contacts 904 and a detector circuit configured to measure signals, e.g., voltage differential signals between pairs of electrical contacts 904 or current flowing between pairs of electrical contacts 904. The controller 906 may further include analog indicators that may provide indications of tissue presence and placement to the user. For example, the controller 906 may include one or several light emitting devices, such as light emitting diodes (LEDs), that may visually indicate to the user regions in which tissue was detected in end effector 912. In an embodiment, respective LEDs may be provided to correspond with the respective ones of the electrical contacts 904, and the controller 906 may be configured to cause the respective LEDs to light up (e.g., green or red) to indicate whether tissue presence was detected in the corresponding region of the end effector 912. In another example, a single light emitting device may be provided, and the controller 906 may be configured to cause the single light emitting device to, for example, light up (e.g., green) in response to detecting that tissue is present in the target region 910 and the tissue that is present in the target region 910 is entirely contained within the target region 910. In another embodiment, an audio indication may be provided to the user in addition to or instead of visual indication.

In some implementations, the controller 906 may be at least partially digital. In some examples, the controller 906 may be implemented utilizing dedicated hardware, such as one or more of discrete components, an integrated circuit, an application-specific integrated circuit (ASIC), a programmable logic device (PLD), a processor executing firmware instructions, a processor executing software instructions, or any combination thereof. When implemented utilizing a processor executing software or firmware instructions, the software or firmware instructions may be stored in any suitable computer readable memory such as on a magnetic disk, an optical disk, or other storage medium, etc. The software or firmware instructions may include machine readable instructions that, when executed by one or more processors, cause the one or more processors to perform various acts related to generating excitation signals to be provided to the sensor circuit 902, to detect tissue characteristics based on measurements obtained using the sensor circuit 902, to detect presence and placement of measurements obtained using the sensor circuit 902, to provide indications to a user of surgical instrument 10, etc. In some implementations, the controller 906 may include one or more digital to analog converters (DACs) and one or more analog to digital converters (ADCs) configured to convert signals between analog signals suitable for use with the sensor circuit 902 and digital signals used with digital circuitry of the controller 906.

In various embodiments, the sensor circuit 902 is configured with only a limited (e.g., minimal) number of electrical contacts 904 that provides useful information (e.g., positioning information) regarding tissue clamped between the jaws of the end effector 912 to the user of the surgical instrument 10. In at least some cases, there may be limited amount of space to position electrical contacts 904 in the end effector 912 and/or to run the conductors 908 to couple the electrical contacts 904 to the controller 906 or to wirelessly transmit signals between the sensor circuit 902 and the controller 906. For example, the controller 906 may be disposed in a portion of the surgical instrument 10 that is external to the end effector 912, such as in the handle portion 20 of the surgical instrument 10. In this case, in an example in which wired connections between the electrical contacts 904 and the controller 906 are provided via the conductors 908, the conductors 908 may run through a limited space or cavity inside the shaft 22 that connects the handle portion 20 to the end effector 912.

As another example, in an example in which a wireless connection is provided between the sensor circuit 902 and the controller 906, the sensor circuit 902 may include additional circuitry for wireless transmission of signals from electrical contacts 904 to the controller 906. Thus, limiting or minimizing the number of electrical contacts 904 may result in a reduced space and power needed to include such circuitry in the end effector 912. Also, with limited or minimized number of electrical contacts 904 in the end effector 912, fewer wireless signals need to be transmitted to and from the end effector 912 in the body of the patient and thus chances of successful transmission may be increased.

As described in more detail below, a minimal arrangement of the electrical contacts 904 in the sensor circuit 902 may include one or more target electrical contacts 904 positioned within the target region 910 of the end effector 912 and one or more external electrical contacts 904 positioned externally adjacent to the target region 910 of the end effector 912 at either one of, or both of, proximal end and distal end of the target region 910 of the end effector 912. The one or more target electrical contacts 904 may include a proximal target electrical contact 904 that extends longitudinally in a proximal half of the target region 910 and a distal target electrical contact 904 that extends longitudinally in a distal half of the target region 910. The proximal target electrical contact 904 is configured to provide a signal indicative of whether tissue is present in the proximal half of the target region 910 and the distal target electrical contact 904 is configured to provide a signal indicative of whether tissue is present in the distal half of the target region 910. In another example, the proximal target electrical contact 904 and the distal target electrical contact 904 may be used to detect whether tissue is present within the target region 910 and whether the tissue that is present within the target region 910 is relatively well-centered within the target region 910, or is proximally or distally shifted within the target region 910. In some examples, the one or more target electrical contacts 904 may include only a single target electrical contact 904 that extends longitudinally between the proximal end and the distal end of the target region 910 and is configured to detect presence or absence of tissue in the target region 910.

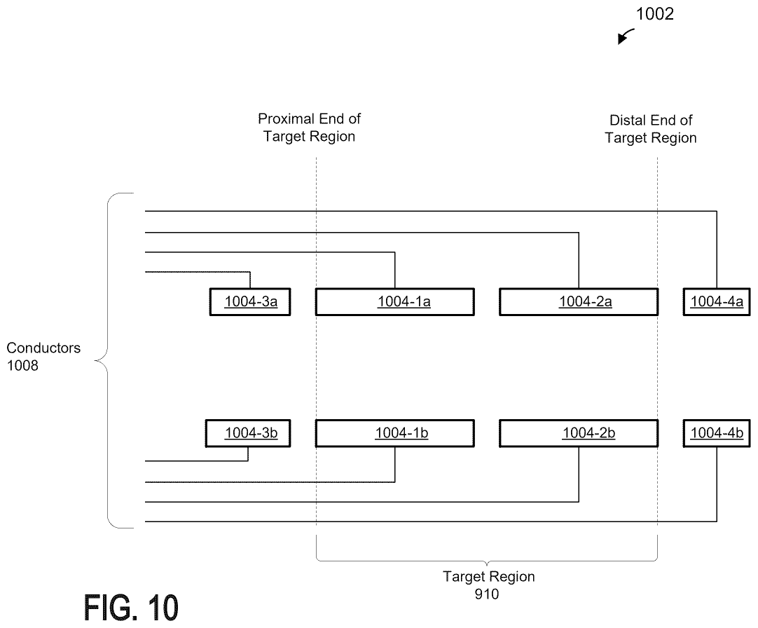

The one or more external electrical contacts 904 may include a distal external electrical contact 904 that is positioned externally adjacent to the distal end of the target region 910. The distal external electrical contact 904 may be configured to provide a signal indicative of whether tissue extends beyond the target region 910. Additionally or alternatively, the one or more external electrical contacts 904 may include a proximal external electrical contact 904 positioned externally adjacent to the proximal end of the target region 910. The proximal external electrical contact 904 may be configured to provide a signal indicative of whether tissue is bunched up in the proximal end of the end effector 912. In some cases, the distal external electrical contact 904 and the proximal external electrical contact 904 may be electrically connected with one another such that a single sensor signal may indicate whether tissue extends beyond the target region 910. These and other minimal arrangements of electrical contacts 904, according to various examples, are described in more detail below in connection with FIGS. 10-13.

As described above, in various embodiments, the controller 906 is configured to provide one or more indications indicating one or both of i) whether tissue is present in the target region and ii) whether the tissue that is present in the target region is contained entirely within the target region to a user (e.g., clinician or robot) of the surgical instrument 10. For example, depending on the number and arrangement of the electrical contacts 904 provided in the sensor circuit 902, the controller 906 may provide indications of one or more of i) whether tissue is present between the jaws of the end effector 912, ii) whether the tissue that is present between the jaws of the end effector 912 is contained entirely within the jaws of the end effector 912, iii) whether tissue that is present between the jaws of the end effector 912 is well-centered within the target region 910 of the end effector 912 or is distally or proximally shifted in the target region 910, iv) one or more characteristics of the tissue that is present between the jaws of the end effector 912 (e.g., whether the tissue has cancerous elements or fatty deposits), v) whether tissue that is present between the jaws of the end effector 912 extends distally beyond the end effector 912, and vi) whether the tissue that is present between the jaws of the end effector 912 is bunched up in the proximal end of the end effector 912.

In various examples, the one or more indications may be provided to the user via one or more of i) visual indicators, such as LEDs, that may be provided, for example, on a handle of the surgical instrument 10, ii) audio indicators and/or iii) a digital screen or other display that may be integrated with the surgical instrument 10 or communicatively coupled to the surgical instrument 10. For example, a transmitter may be coupled to the controller 906 to wirelessly transmit tissue presence and/or placement signals generated using the plurality of electrical contacts 904 to a processor of a computer that may be located, for example, in an operating room in which the surgical instrument 10 is being used. The processor of the computer may be configured to further process the tissue presence and/or placement signals and/or cause an indication of tissue presence and/or placement, determined based on the tissue presence and/or placement signals, to be displayed on a display coupled to the computer. In other embodiments, other suitable indicators may be utilized.

FIG. 10 is a diagram depicting a sensor circuit 1002 that may be used with an end effector such as the end effector 912 of FIG. 9, according to an embodiment. In an embodiment, the sensor circuit 1002 corresponds to the sensor circuit 902 of the end effector 912 of FIG. 9. The sensor circuit 1002 is described with reference to FIG. 9 for ease of explanation. In other embodiments, the sensor circuit 1002 is used with end effectors different from the end effector 912 of FIG. 9. Similarly, the sensor circuit 902 of the end effector 912 of FIG. 9 is different from the sensor circuit 902, in some embodiments.

The sensor circuit 1002 includes a plurality of electrical contacts 1004 that correspond to the electrical contacts 904. In the configuration of FIG. 10, the electrical contacts 1004 are configured as electrical contact pairs, and the electrical contacts 1004 are sometimes referred to herein as electrical contact pairs 1004. Each electrical contact pair 1004 includes i) a source electrical contact a that serves as a source to which an excitation signal is applied and ii) a reference or return electrical contact b that is used as a reference for measuring a voltage differential, current flow, capacitance, inductance, etc. when the excitation signal is provided to the source electrical contact a. The electrical contact pairs 1004 include target electrical contact pairs positioned within the target region 910 of the end effector 912 and external electrical contact pairs positioned externally adjacent to the target region 910 of the end effector 912. The target electrical contact pairs 1004 include a proximal target electrical contact pair 1004-1 that extends in a proximal half the target region 910 and a distal target electrical contact pair 1004-2 that extends in a distal half of the target region 910. The external electrical contact pairs 1004 include a proximal external contact pair 1004-3 positioned externally adjacent to a proximal end of the target region 910 and a distal external contact pair 1004-4 positioned externally adjacent to a distal end of the target region 910.

The external electrical contact pairs 1004-3, 1004-4 may be positioned just outside of the target region 910 along the length of the end effector 912, each external electrical contact positioned such that there is a small gap between the external electrical contact and a corresponding target electrical contact that is positioned within the target region 910. The gaps between an external electrical contact and a corresponding target electrical contact that is positioned within the target region 910 may be sized to be just large enough to prevent issues with interference or “cross talking” between the external electrical contact and the corresponding target electrical contact that is positioned within the target region 910. Thus, for example, the electrical contacts of the proximal external electrical pair 1004-3 may be positioned just beyond the proximal end of the target region 910 along the length of the end effector 912, in the direction from the proximal end of the target region 910 towards the proximal end of the end effector 912 with a small (e.g., 0.5 mm to 1.5 mm) gap between each electrical contact of the proximal external electrical pair 1004-3 and the corresponding electrical contact of the proximal electrical contact target pair 1004-1. Similarly, the electrical contacts of the distal external electrical pair 1004-4 may be positioned just beyond the distal end of the target region 910 along the length of the end effector 912, in the direction from the distal end of the target region 910 towards the distal end of the end effector 912, with a small (e.g., 0.5 mm to 1.5 mm) gap between each electrical contact of the distal external electrical pair 1004-4 and the corresponding electrical contact of the distal target electrical contact pair 1004-2.

The electrical contacts of the proximal target electrical contact pair 1004-1 and the distal target electrical contact pair 1004-2 may be sized to collectively span the length of the target region 910 with a small gap between the proximal target electrical contact pair 1004-1 and the distal target electrical contact pair 1004-2. For example, in an embodiment in which the target region covers approximately 80% of the end effector 912 along the length of the end effector 912, each electrical contact of the proximal target electrical contact pair 1004-1 and the proximal target electrical contact pair 1004-1 may span just under 40% (e.g., 39% or 38%) of the end effector 912 along the length of the end effector 912, such that there is a small gap between the proximal target electrical contact pair 1004-1 and the distal target electrical contact pair 1004-2. Thus, for example, in an embodiment in which the length of the end effector 912 is approximately 60 mm, each electrical contact of the proximal target electrical contact pair 1004-1 and the distal target electrical contact pair 1004-2 may span just under 30 mm (e.g., 29 mm or 28 mm), such that there is a small (e.g., 0.5 mm to 1.5 mm) gap between the proximal target electrical contact pair 1004-1 and the distal target electrical contact pair 1004-2. The gap between the proximal target electrical contact pair 1004-1 and the distal target electrical contact pair 1004-2 may be sized to be just large enough to prevent issues with interference or “cross talking” between the proximal target electrical contact pair 1004-1 and the distal target electrical contact pair 1004-2.

In some examples, the electrical contacts of the external electrical contact pairs 1004-3, 1004-4 may be relatively shorter as compared to the electrical contacts of the target electrical contact pairs 1004-1, 1004-2. In other words, respective ones of the one or more target electrical contacts of the target electrical contact pairs 1004-1, 1004-2 are relatively greater in length as compared to respective ones of the one or more external electrical contacts of the external electrical contact pairs 1004-3, 1004-4. In an example, the electrical contacts of the external electrical contact pairs 1004-3, 1004-4 may be sized just large enough to have electrical properties of a pad rather than just a wire, so that tissue contact can be properly measured using the external electrical contacts 1004-3, 1004-4. In at least some embodiments, the small size of the electrical contacts of the external electrical contact pairs 1004-3, 1004-4 may ensure that these electrical contacts will only sense when tissue that is present within the target region 910 extends beyond the respective ends of the target region 910, and will not sense any other tissue that may be present outside of the target region 910, for example. In an example, each electrical contact of the external electrical contact pairs 1004-3, 1004-4 may be approximately 0.5 mm to 0.75 mm in length. In another example, each electrical contact of the external electrical contact pairs 1004-3, 1004-4 may be approximately 1.5 mm in length. In other examples, other suitable small electrical contacts may be utilized.

The controller 906 may be configured to apply an excitation signal to the source electrical contact a of an electrical contact pair 1004, and to measure a response signal between the source electrical contact a and the reference electrical contact b of the electrical contact pair 1004. For example, the controller 906 may be configured to apply an AC current signal to the source electrical contact a of the electrical contact pair 1004 and to measure a resulting voltage differential between the source electrical contact a and the reference electrical contact b of the electrical contact pair 1004. As another example, the controller 906 may be configured to apply an AC voltage signal to the source electrical contact a of the electrical contact pair 1004 and to measure a resulting current flowing between the source electrical contact a and the reference electrical contact b of the electrical contact pair 1004.

The controller 906 is configured to, based on the measurements performed using the electrical contacts 1004, detect presence and positioning of tissue between the jaws of the end effector 912. For example, the controller 906 may be configured to detect, based on measurements obtained using the target electrical contact pairs 1004, whether tissue is present in the target region 910 of the end effector 912. The tissue characteristic (e.g., impedance, capacitance, inductance, etc.) measured using a particular source electrical contact 1004 may indicate whether the particular source electrical contact 1004 is in contact with (or in sufficient proximity to) tissue or is in contact with (or in sufficient proximity to) a substance other than tissue (e.g., blood). In this way, the controller 906 is configured to detect the tissue presence and placement between the lower jaw 16 and the upper jaw 18 of the end effector 912 based on measurements of tissue impedance in the target region 910 and externally adjacent to the target region 910.

The controller 906 may be configured to detect whether a particular source electrical contact 1004 is in contact with tissue based on monitoring changes the tissue characteristic (e.g., impedance, capacitance, inductance, etc.) measured using the particular source electrical contact 1004 as the end effector clamps down, and determining that the particular source electrical contact 1004 comes in contact with the tissue if the change (e.g., increase) of the particular tissue characteristic exceeds a predetermined threshold. In another example, the controller 906 may be configured to detect whether a particular source electrical contact 1004 is in contact with tissue by detecting whether the tissue characteristic measured using the particular source electrical contact 1004 when the end effector 912 clamps down exceeds a predetermined threshold.

The controller 906 may be configured to determine, based on detecting whether respective ones of the source electrical contacts 1004 are in contact with tissue, i) whether tissue is present in the target region 910 of the end effector 912 and ii) whether the tissue that is present in the target region 910 is entirely contained within the target region 910 of the end effector 912. For example, based on detecting that none of the source electrical contacts 1004 are in contact with tissue, the controller 906 may determine that no tissue is present within the jaws of the end effector. As another example, based on detecting that the source electrical contact 1004-1a of the proximal target electrical contact pair 1004-1 and/or the source electrical contact 1004-2a of the distal target electrical contact pair 1004-2 is in contact with tissue, the controller 906 may determine that tissue is present in the target region 910 between the jaws of the end effector 912. Further, based on detecting that both the source electrical contact 1004-3a of the proximal external electrical contact pair 1004-3 and the source electrical contact 1004-4a of the distal external electrical contact pair 1004-4 are not in contact with tissue, the controller 906 may determine that the tissue that is present in the target region 910 between the jaws of the end effector 912 is contained entirely within the target region 910.

On the other hand, based on detecting that i) the source electrical contact 1004-1a of the proximal target electrical contact pair 1004-1 and/or the source electrical contact 1004-2a of the distal target electrical contact pair 1004-2 is in contact with tissue and ii) the source electrical contact 1004-3a and/or the source electrical contact 1004-4a is in contact with tissue, the controller 906 may determine that tissue is present in the target region 910 between the jaws of the end effector 912 and that the tissue is not entirely contained within the target region 910 of the end effector 912. For example, based on detecting that the source electrical contact 1004-4a is in contact with tissue, the controller 906 may determine that the tissue extends distally outside of the target region 910. Presence of tissue at the location of the source electrical contact 1004-4a may indicate that the tissue may not be cut in its entirety with a single firing of the end effector 912. As another example, based on detecting that the source electrical contact 1004-3a is in contact with tissue, the controller 906 may determine that the tissue extends proximally outside of the target region 910. Presence of tissue at the location of the source electrical contact 1004-3a may indicate that the tissue is gathered or bunched up in the proximal end of the end effector 912 and, thus, may lead to sub-optimal stapling of the tissue by the end effector 912.

As yet another example, based on detecting that i) the source electrical contact 1004-1a of the proximal target electrical contact pair 1004-1 and the source electrical contact 1004-2a of the distal target electrical contact pair 1004-2 are both not in contact with tissue and ii) the source electrical contact 1004-3a and/or the source electrical contact 1004-4a is in contact with tissue, the controller 906 may determine that tissue is present only outside of the target region 910 of the end effector 912 (e.g., only outside of the proximal end of the target region 910 and/or only outside of the distal end of the target region 910).

In some embodiments, the controller 906 may also be configured to detect whether tissue that is present in the target region 910 is relatively well centered within the target region 910. For example, based on detecting that both the source electrical contact 1004-1a in the distal half of the target region 910 and the source electrical contact 1004-2a in the proximal half of the target region 910 are in contact with tissue, the controller 906 may determine that the tissue is relatively well centered in the target region 910. On the other hand, based on detecting that one of the source electrical contact 1004-1a and the source electrical contact 1004-2a is in contact with tissue, but the other one of the source electrical contact 1004-1a and the source electrical contact 1004-2a is not in contact with tissue, the controller 906 may determine that the tissue is not well centered in the target region 910. For example, based on detecting that the source electrical contact 1004-1a is in contact with tissue whereas the source electrical contact 1004-2a is not in contact with tissue, the controller 906 may determine that the tissue that is present between the jaws of the end effector 912 is shifted towards the proximal end of the end effector 912. As another example, based on detecting that the source electrical contact 1004-2a is in contact with tissue whereas the source electrical contact 1004-1a is not in contact with tissue, the controller 906 may determine that the tissue that is present between the jaws of the end effector 912 is shifted towards the distal end of the end effector 912.

It is noted that although example tissue presence and placement scenarios are described herein, these examples are not exhaustive. Other tissue present and placement scenarios may be detected, in other embodiments. As just an example, based on detecting that i) the electrical contact 1004-1a of the proximal target electrical contact pair 1004-1 is in contact with tissue, ii) the source electrical contact 1004-2a of the distal target electrical contact pair 1004-2 is not in contact with tissue, iii) the source electrical contact 1004-3a is not in contact with tissue, and iv) the source electrical contact 1004-4a is in contact with tissue, the controller 906 may determine that tissue is present in the proximal half of the target range 910 and tissue is also present outside the distal end of the target range 910, while no tissue is present in the distal half of the target region 910 or outside of the proximal end of the target region 910. In other examples, the controller 906 may determine other tissue presence and placement scenarios based on detecting which of the source electrical contacts 104, if any, are in contact with tissue.

As described above, in various embodiments, the controller 906 is configured to provide one or more indications of the detected tissue presence and placement between the jaws of the end effector 912 to the user of the surgical instrument 10 to enable the user to make appropriate decisions prior to firing of the end effector 912. Such indications may be provided to the user via visual indicators disposed, for example, on the handle of the surgical instrument 10, via a display (e.g., a screen) disposed on or otherwise coupled to the surgical instrument 10, via an auditory output of the surgical instrument 10, etc., in various embodiments. In an embodiment, using the arrangement of electrical contacts illustrated in FIG. 10, the one or more indications provided by the controller 906 may include indications of one or more of i) whether tissue is present between the jaws of the end effector 912, ii) whether the tissue that is present between the jaws of the end effector 912 is contained entirely within the jaws of the end effector 912, iii) whether tissue that is present between the jaws of the end effector 912 is well-centered within the target region 910 of the end effector 912 or is distally or proximally shifted in the target region 910, iv) one or more characteristics of the tissue that is present between the jaws of the end effector 912 (e.g., whether the tissue has cancerous elements or fatty deposits), v) whether tissue that is present between the jaws of the end effector 912 extends distally beyond the end effector 912, and vi) whether the tissue that is present between the jaws of the end effector 912 is bunched up in the proximal end of the end effector 912. Such indications may allow the user to make appropriate adjustments, such as repositioning of the end effector 912, prior to firing and may, in at least some scenarios, lead to cost savings and/or better surgical outcomes for the patient.

In some embodiments, a sensor circuit similar to the sensor circuit 1002, but including a greater or a smaller number of electrical contacts as compared to the number of the electrical contacts 1004 of the sensor circuit 1002, may be used. For example, a greater number of electrical contacts may be placed in the target region of the end effector to increase granularity of detection of tissue placement within the target region of the end effector. Generally, however, in various embodiments, the number of electrical contacts is relatively small due to limited space on the end effector and/or limited space for routing electrical connections out of the end effector and running conductors (e.g., wires) to connect the electrical elements to a controller external to the end effector. Further, while more electrical contacts may lead to better overall resolution, more electrical contacts is more expensive and may be cost prohibitive.

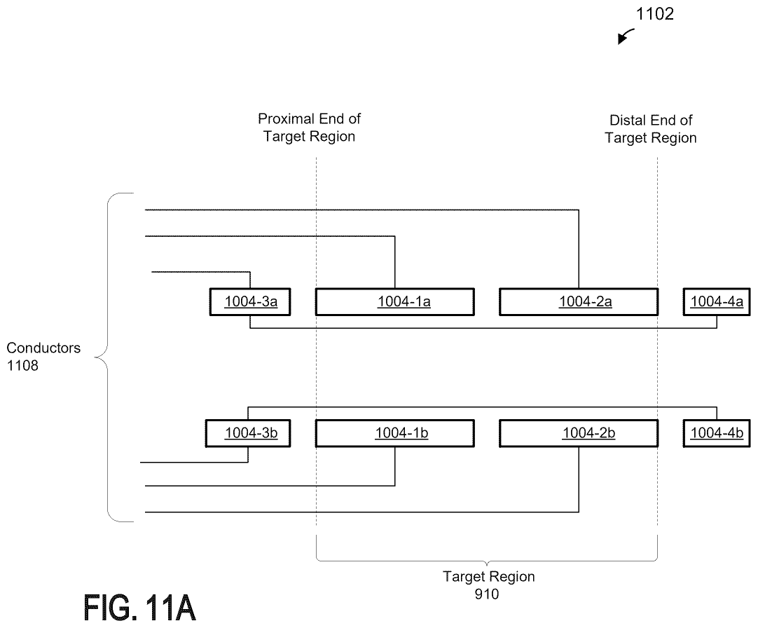

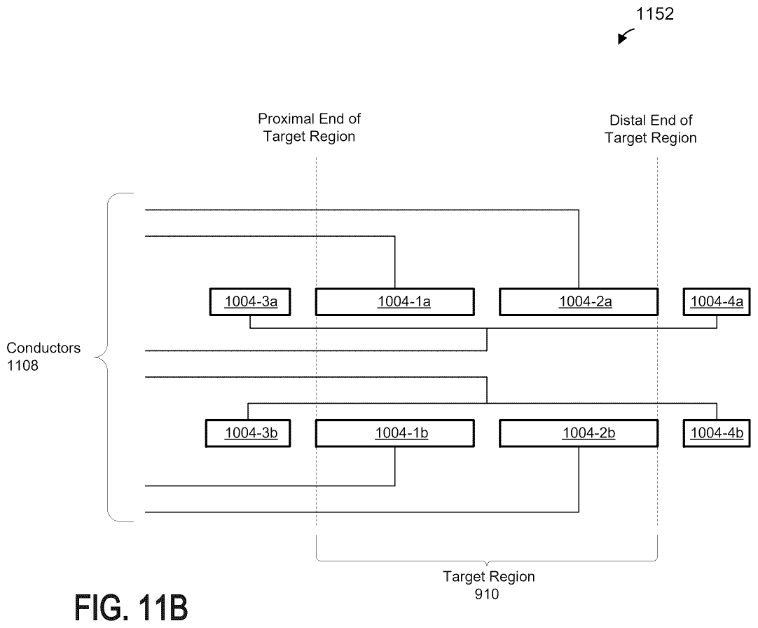

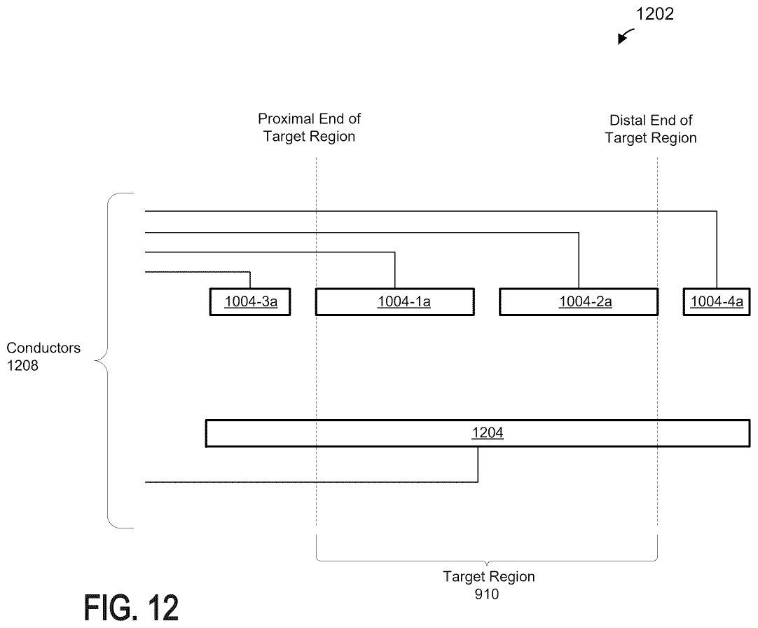

As another example, in some embodiments, the number of electrical contacts of a sensor circuit and/or the number of conductors (e.g., wires) needed to connect the electrical elements to a controller external to the end effector may be reduced as compared to the number of the electrical contacts 1004 of the sensor circuit 1002. For example, one or more electrical contacts placed in the end effector may be connected together so that a single conductor is needed to connect multiple electrically connected contacts to the controller external to the end effector. As another example, some electrical contacts 1004 may be replaced with a single electrical contact to reduce the number of conductors needed to connect the electrical contacts to the controller, at the expense of detection granularity, for example. Several example sensor circuits with reduced numbers of electrical contacts and conductors needed to connect the electrical contacts to the controller, according to some embodiments, are described briefly below with reference to FIGS. 11 and 12.

FIGS. 11A-B are diagrams of, respectively, sensor circuits 1102, 1152 with a reduced number of signals needed for detection, according to embodiments. In embodiments, the sensor circuits 1102, 1152 correspond to the sensor circuit 902 of the end effector 912 of FIG. 9. The sensor circuits 1102, 1152 are described with reference to FIG. 9 for ease of explanation. In other embodiments, the sensor circuits 1102, 1152 are used with end effectors different from the end effector 912 of FIG. 9. Similarly, the sensor circuit 902 of the end effector 912 of FIG. 9 is different from the sensor circuits 1102, 1152, in some embodiments.

The sensor circuits 1102, 1152 are generally similar to the sensor circuit 1002 of FIG. 10 and include same-numbered elements with the sensor circuit 1002 of FIG. 10. However, in the sensor circuits 1102, 1152, the external electrical contacts 1004 are electrically connected with each other to reduce the number of conductors needed to connect the electrical contacts 1004 to the controller 906. In particular, in the embodiment of FIG. 11A, the external source electrical contact 1004-3a and the external source electrical contact 1004-4a are electrically connected together in a serial connection in the sensor circuit 1102. Similarly, the external reference electrical contact 1004-3b and the external source electrical contact 1004-4b are electrically connected together in a serial connection the sensor circuit 1102. In the embodiment of FIG. 11B, the external source electrical contact 1004-3a and the external source electrical contact 1004-4a are electrically connected together in parallel in the sensor circuit 1152. Similarly, the external reference electrical contact 1004-3b and the external source electrical contact 1004-4b are electrically connected together in parallel in sensor circuit 1152. In other examples, the external reference electrical contact 1004-3b and the external source electrical contact 1004-4b may be electrically connected together in other suitable manners.

Because the external reference electrical contact 1004-3b and the external source electrical contact 1004-4b are electrically connected together in the sensor circuits 1102, 1152, only three pairs of conductors 1108 are needed to connect the electrical contacts 1004 to the controller 906. Thus, in these examples, the controller 906 is coupled to the electrical contacts 1004 using a number of connections less than a number of electrical contacts among the electrical contacts 1004. As mentioned above, while this simplifies the sensor circuit and may reduce costs, the resolution or granularity of detection is slightly decreased, since a single signal provided by the electrically connected external electrical contacts 1004 only indicates that tissue is present outside the target region 910, but does not indicate whether the tissue extends distally beyond the distal end of the target region 910 or whether the tissue is bunched up on the proximal end of the end effector 912, as it would if the sensor circuit were providing separate signals from each of the external electrical contacts 1004-3, 1004-4, as discussed above. In some examples, electrical contacts of an end effector may be interconnects in other suitable manners to provide a sensor arrangement in which the controller 906 is coupled to the electrical contacts using a number of connections less than a number of electrical contacts among the electrical contacts.

In an embodiment, with the arrangement of electrical contacts 1004 in the sensor circuits 1102, 1152, the controller 906 may be configured to detect, and inform a user of, one or more of i) whether tissue is present in the target region 910 of the end effector 912, ii) whether the tissue that is present in the target region 910 is well-centered, or distally or proximally shifted, in the target region 910 of the end effector 912, iii) one or more characteristics of the tissue that is present in the target region 910 of the end effector 912 (e.g., whether the tissue has cancerous elements or fatty deposits), and iv) whether the tissue that is present in the target region 910 is contained entirely within the target region 910. In other examples, the controller 906 may be additionally or alternatively configured to detect, and inform the user of, other tissue placement information that is detectable using the arrangement of electrical contacts 1004 in the sensor circuits 1102, 1152.

FIG. 12 is a diagram of a sensor circuit 1202 with a reduced number of refence electrical contacts, according to an embodiment. In an embodiment, the sensor circuit 1202 corresponds to the sensor circuit 902 of the end effector 912 of FIG. 9. The sensor circuit 1202 is described with reference to FIG. 9 for ease of explanation. In other embodiments, the sensor circuit 1202 is used with end effectors different from the end effector 912 of FIG. 9. Similarly, the sensor circuit 902 of the end effector 912 of FIG. 9 is different from the sensor circuit 1202, in some embodiments.

The sensor circuit 1202 is generally similar to the sensor circuit 1002 of FIG. 10 and includes like-numbered elements with the sensor circuit 1002 of FIG. 10. However, in the sensor circuit 1202, the multiple reference electrode contacts b are replaced with a single reference electrical contact 1204. With the multiple reference electrode contacts b replaced with the single electrical contact 1204, a single conductor 1208 is needed to connect the single electrical contact 1204 to the controller 906. Thus, the number of conductors 1208 needed to connect the electrical contacts of the sensor circuit 1202 to the controller 906 is reduced as compared to the number of conductors 1008 in FIG. 10. In an embodiment, with the arrangement of electrical contacts 1004, 1204 in the sensor circuit 1202, the controller 906 may be configured to sequentially query the source electrical contacts 1004-1a, 1004-2a, 1004-3a, 1004-4a one by one to obtain tissue characteristic measurements and detect presence and placement of tissue using the electrical contacts 1004, 1204. It is noted that although the sensor circuit 1202 is illustrated as including a single reference electrical contact 1204 used with a single conductor 1208, the sensor circuit 1202 may include two or three separate reference electrical contacts 1204 used with a corresponding number of separate conductors 1208, in some embodiments. For example, a first reference electrical contact 1204 and a first conductor 1208 may be used with the target electrical contacts 1004-1a and 1004-2a to detect whether tissue is present in the target region 910, and a separate second reference electrical contact 1204 (or, alternatively two separate electrically connected electrical contacts 1204) and a second conductor 1208 may be used with the external electrical contacts 1004-3a and 1004-4a to detect whether the tissue that is present in the target region 910 is contained entirely within the target range 910, in an embodiment. It is also noted that although the reduced reference electrical contact configurations are generally described with reference to the sensor circuit 1202 of FIG. 12, such reduced reference electrical contact configurations may be utilized with other sensor circuit configurations, in some embodiments. For example, the sensor circuit 1102 of FIG. 11A or the sensor circuit 1152 of FIG. 11B may have a reduced reference electrical conductor configuration, in some embodiments.

In one embodiment, the proximal target electrical contact pair 1004-1 of FIG. 9, 10 or 11A-B or the source electrical contact 1004-1a (i.e., proximal source electrical contact 1004-1a) of FIG. 12 may be shorter in length than the distal target electrical contact pair 1004-2 of FIG. 9, 10, or 11A-B and the source electrical contact 1004-2a (i.e., distal source electrical contact 1004-2a) of FIG. 12, respectively. In this example, the distal target electrical contact pair 1004-2 of FIG. 9, 10 or 11A-B and the distal source electrical contact 1004-2a of FIG. 12 may extend substantially the entire length of the target region 910. In this configuration, the proximal target electrical contact pair 1004-1 of FIG. 9, 10 or 11A-B or the proximal source electrical contact 1004-1a of FIG. 12 act as a proximal or “bunch” sensor, indicating when tissue is present close to the proximal end of the end effector. In this scenario, the user may decide to adjust the end effector so that the grasped tissue is only read by the distal target electrical contact pair 1004-2 of FIG. 9, 10 or 11A-B or the distal source electrical contact 1004-2a of FIG. 12 to avoid the situation in which the tissue is too close to the proximal end of the end effector, for example even when the external electrical contacts 1004 do not indicate that the tissue extends proximally outside of the target region 910 of the end effector.

In yet another embodiment, the reverse of the above may be true, where the distal target electrical contact pair 1004-1 of FIG. 9, 10 or 11A-B or the source electrical contact 1004-2a (i.e., distal source electrical contact 1004-2a) of FIG. 12 may be shorter in length than the proximal target electrical contact pair 1004-1 of FIG. 9, 10 or 11A-B and the source electrical contact 1004-1a (i.e., proximal source electrical contact 1004-1a) of FIG. 12, respectively. In this example, the proximal target electrical contact pair 1004-1 of FIG. 9, 10 or 11A-B and the proximal source electrical contact 1004-1a of FIG. 12 may extend substantially the entire length of the target region 910. In this configuration, the distal target electrical contact pair 1004-2 of FIG. 9, 10 or 11A-B or the distal source electrical contact 1004-2a of FIG. 12 act as a distal or “extension” sensor, indicating when tissue is present close to the distal end of the end effector. In this scenario, the user may decide to adjust the end effector so that the grasped tissue is only read by the proximal target electrical contact pair 1004-1 of FIG. 9, 10 or 11A-B or the proximal source electrical contact 1004-1a of FIG. 12 to avoid the situations in which the tissue is too close to the distal end of the end effector, for example even when the external electrical contacts 1004 do not indicate that the tissue extends distally outside of the target region 910 of the end effector.

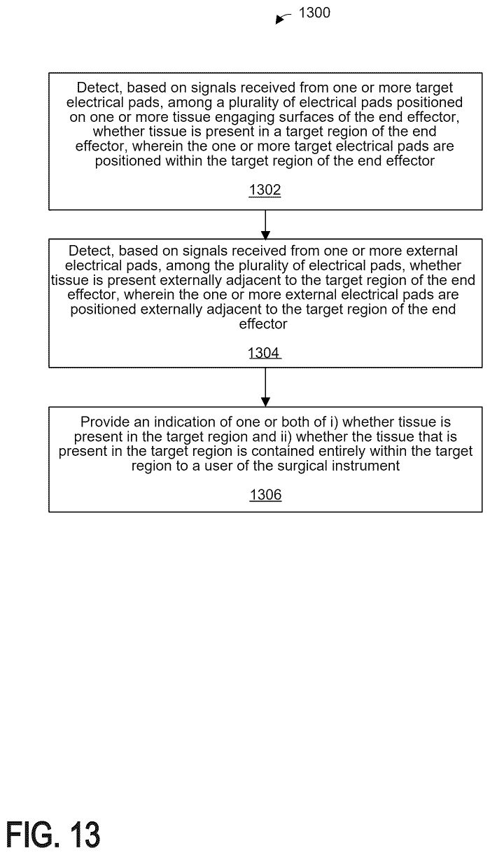

FIG. 13 is a flowchart of a method 1300 for determining presence and placement of tissue grasped between a first jaw and a second jaw of an end effector of a surgical instrument, according to an embodiment. In an embodiment, the method 1300 is implemented by the controller 906 to determine presence and placement of tissue grasped between the lower jaw 16 and the upper jaw 18 of the end effector 912 of FIG. 9 used with the surgical instrument 10 of FIG. 1. It is noted, however, that the method 1300 may be implemented by a controller different from the controller 906 and/or with an end effector different from the end effector 912 and/or with a surgical instrument different from the surgical instrument 10, in other embodiments.

At a block 1302, the controller 906 may detect, based on signals received from one or more target electrical pads, among a plurality of electrical pads positioned on one or more tissue engaging surfaces of the end effector, whether tissue is present in a target region of the end effector. The one or more target electrical pads may be positioned within the target region of the end effector, and the controller 906 may be configured to detect whether tissue is present in the target region of the end effector by detecting whether the one or more target electrical pads are in contact with (or in sufficient proximity to) tissue in the end effector. The plurality of electrical pads may comprise passive electrical components, such as electrodes or other conductive or non-conductive electrical pads, in an embodiment. In another embodiments, active sensors may be used in addition to or instead of the passive electrical components.

The one or more target electrical pads may include a distal target electrical pad extending longitudinally in a distal half of the target region 910 and a proximal target electrical pad extending longitudinally in a proximal half of the target region 910. In another example, the one or more target electrical pads may include a single target electrical pad extending longitudinally in the target region 910. In other examples, the one or more target electrical pads include a greater number of electrical pads to increase granularity of tissue placement measurements. However, the number of target electrical pads may be kept relatively small to reduce or minimize the number of electrical pads and/or the number of signals and/or conductors (e.g., wires) that need to be provided between the controller 906 and the electrical pads in the end effector 912.

In an embodiment, the controller 906 is configured to detect whether tissue is present in the target region of the end effector by performing tissue impedance measurements using the one or more target electrical pads. In other embodiments, the controller 906 may be configured to detect whether tissue is present in the target region of the end effector by performing other measurements (e.g., tissue capacitance measurements, tissue inductance measurements, etc.).

At a block 1304, the controller 906 may detect, based on signals received from one or more external electrical pads, among the plurality of electrical pads, whether tissue is present externally adjacent to the target region of the end effector. The one or more external electrical pads may be positioned externally adjacent to the target region of the end effector, and the controller 906 may be configured to detect whether tissue is present externally adjacent to the target region by detecting whether the one or more external electrical pads are in contact with (or in sufficient proximity to) tissue in or near the end effector. The one or more external electrical pads may include a proximal external electrical pad positioned just beyond the proximal end of the target region 910 along the length of the end effector 912, in the direction from the proximal end of the target region 910 towards the proximal end of the end effector 912. Additionally, or alternatively, the one or more external electrical pads may include a distal external electrical pad positioned just beyond the distal end of the target region 910 along the length of the end effector 912, in the direction from the distal end of the target region 910 towards the distal end of the end effector 912.

In an embodiment, the controller 906 is configured to detect whether tissue is present externally adjacent to the target region by performing tissue impedance measurements using the one or more target electrical pads. In other embodiments, the controller 906 may be configured to detect whether tissue is present externally adjacent to the parget region by performing other measurements (e.g., tissue capacitance measurements, tissue inductance measurement, etc.).

At a block 1306, the controller 906 may provide, based on the determination, at block 1302, of whether tissue is present in the target region of the end effector and the determination, at block 1304, of whether tissue is present externally adjacent to the target region of the end effector, one or more indications indicating one or both of i) whether tissue is present in the target region and ii) whether the tissue that is present in the target region is contained entirely within the target region to a user of the surgical instrument. In some embodiments, the controller 906 may additionally or alternatively provide other indications determined based on measurements performed using the one or more target electrical pads and/or the one or more external electrical pads among the plurality of electrical pads. In various examples, depending on the number of target electrical pads and the number of external electrical pads provided in the end effector 912, the controller 906 may provide indications of one or more of i) whether tissue is present between the jaws of the end effector 912, ii) whether the tissue that is present between the jaws of the end effector 912 is contained entirely within the jaws of the end effector 912, iii) whether tissue that is present between the jaws of the end effector 912 is well-centered within the target region 910 of the end effector 912 or is distally or proximally shifted in the target region 910, iv) one or more characteristics of the tissue that is present between the jaws of the end effector 912 (e.g., whether the tissue has cancerous elements or fatty deposits), v) whether tissue that is present between the jaws of the end effector 912 extends distally beyond the end effector 912, and vi) whether the tissue that is present between the jaws of the end effector 912 is bunched up in the proximal end of the end effector 912.

In various examples, the one or more indications may be provided to the user via one or more of i) visual indicators, such as LEDs, that may be provided, for example, on a handle of the surgical instrument, ii) audio indicators and/or iii) a digital screen or other display that may be integrated with the surgical instrument or communicatively coupled to the surgical instrument. For example, a transmitter may be coupled to the controller to wirelessly transmit tissue presence and/or placement signals generated using the plurality of electrical pads 904 to a processor of a computer that may be located, for example, in an operating room in which the surgical instrument 10 is being used. The processor of the computer may be configured to further process the tissue presence and/or placement signals and/or cause an indication of tissue presence and/or placement, determined based on the tissue placement signals, to be displayed on a display coupled to the computer. In other embodiments, other suitable indicators may be utilized.

As indicated above, the detecting steps occur as the end effector clamps down to grasp the tissue and the electrical pads come into contact with the tissue. As also indicated above, detecting tissue characteristics may include measuring a change in impedance as the end effector 912 clamps down on the tissue. Measuring this change in impedance as the end effector 912 clamps down on the tissue may include providing an alternating current (AC) signal to the particular electrical pad that comes into pad with the tissue; measuring a voltage difference between the particular electrical pad and a reference electrical pad provided on the end effector 912; and detecting the change in impedance based on the voltage difference.

The embodiments disclosed herein provide electrical contacts (e.g., electrical pads) distributed along one or more jaws of an end effector to show presence and positioning of tissue clamped between the jaws of the end effector to a user while minimizing data output and hardware components. The disclosed embodiments reduce the number of electrical contact/conductor pairs significantly, which enables wireless communication or routing conductors from the end effector “upstream” to the handle. These small and dedicated electrical contact/conductor pairs improve surgical procedures by informing the user of tissue presence in specific areas or regions of the end effector and reducing false assessments of partially clamped tissue.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical instrument (10), comprising:
a shaft (22);
an end effector (12) extending from the shaft (22), the end effector (12) comprising:
   a first jaw (16); and
   a second jaw (18), wherein at least one of the first jaw (16) and the second jaw (18) is movable relative to the other to grasp tissue;
   a plurality of electrical pads (904) positioned, on one or more tissue engaging surfaces of the end effector (12), between a proximal end and a distal end of the end effector (12), the plurality of electrical pads (904) including i) one or more target electrical pads positioned within a target region (910) defined for tissue placement between the first jaw (16) and the second jaw (18) of the end effector (12) and ii) one or more external electrical pads positioned externally adjacent to the target region (910); and
a controller (906) coupled to the plurality of electrical pads (904) so as to receive signals from the electrical pads (904), the controller (906) configured to:
   detect, based on the signals received from the electrical pads (904), tissue presence and positioning between the first jaw (16) and the second jaw (18) of the end effector (12), the controller (906) being configured to detect at least i) whether tissue is present in the target region (910) and ii) whether the tissue that is present in the target region (910) is contained entirely within the target region (910) and not present external to the target region (910); and
   provide an indication of the tissue presence and positioning to a user of the surgical instrument (10).

### Example 2

The surgical instrument (10) of claim Example 1, wherein the one or more target electrical pads positioned within the target region (910) comprise:
a first target electrical pad extending longitudinally through a distal half of the target region (910), the first target electrical pad configured to provide a signal indicative of whether tissue is present in the distal half of the target region (910); and
a second target electrical pad extending longitudinally through a proximal half of the target region (910), the second target electrical pad configured to provide a signal indicative of whether tissue is present in the proximal half of the target region (910).

### Example 3

The surgical instrument (10) of Example 1 or 2, wherein the one or more target electrical pads positioned within the target region (910) comprise a single target electrical pad extending longitudinally between a distal end of the target region (910) and a proximal end of the target region (910).

### Example 4

The surgical instrument (10) of any of Examples 1-3, wherein the one or more external electrical pads positioned externally adjacent to the target region (910) comprise:
a first external electrical pad positioned distally adjacent to the target region (910), the first external electrical pad configured to provide a signal indicative of whether the tissue that is present in the target region (910) extends beyond a distal end of the target region (910); and
a second external electrical pad positioned proximally adjacent to the target region (910), the second external electrical pad configured to provide a signal indicative of whether the tissue that is present in the target region (910) extends beyond a proximal end of the target region (910).

### Example 5

The surgical instrument (10) of Example 4, wherein the first external electrical pad and the second external electrical pad are electrically connected with one another to provide a single signal indicative of whether the tissue that is present in the target region (910) extends beyond a distal end of the end effector (12) or is bunched up at a proximal end of the end effector (12).

### Example 6

The surgical instrument (10) of any of Examples 1-5, wherein respective ones of the one or more target electrical pads are relatively greater in length as compared to respective ones of the one or more external electrical pads.

### Example 7

The surgical instrument (10) of any of Examples 1-6, wherein the controller (906) is coupled to the plurality of electrical pads (904) using a number of connections less than a number of electrical pads among the plurality of electrical pads (904).

### Example 8

The surgical instrument (10) of any of Examples 1-7, wherein the controller (906) is configured to detect the tissue presence and positioning between the first jaw (16) and the second jaw (18) of the end effector (12) based on measurements of tissue impedance in the target region (910) and externally adjacent to the target region (910).

### Example 9

The surgical instrument (10) of any of Examples 1-8, further comprising a staple cartridge installed into a channel in one of the first jaw (16) and the second jaw (18) of the end effector (12), wherein the plurality of electrical pads (904) are disposed on one or both of the staple cartridge and the channel into which the staple cartridge is installed in the end effector (12).

### Example 10

The surgical instrument (10) of any of Examples 1-9, further comprising:
a handle portion (20) connected to the shaft (22), wherein the controller (906) is contained in the handle portion (20); and
a plurality of conductors (908) that run through a cavity in the shaft (22) to couple the plurality of electrical pads (904) in the end effector (12) to the controller (906) contained in the handle portion (20).

### Example 11

The surgical instrument (10) of Example 10, wherein:
the handle portion (20) is equipped with one or more indicators; and
the controller (906) is configured to cause a tissue positioning indication to be provided to the user via the one or more indicators.

### Example 12

The surgical instrument (10) of any of Examples 1-11, further comprising a transmitter coupled to the controller (906), wherein the transmitter is configured to wirelessly transmit tissue positioning signals generated using the plurality of electrical pads (904) to a processor of a computer, the processor being configured to one or both of i) further process the tissue positioning signals or ii) cause the indication of tissue positioning, determined based on the tissue positioning signals, to be displayed on a display coupled to the computer.

### Example 13

The surgical instrument (10) of any one of Examples 1 to 12, wherein the controller is configured to:
detect a tissue characteristic using the one or more target electrical pads as the end effector (12) clamps down to grasp the tissue; and
detect whether tissue is present in the target region (910) based on the tissue characteristic.

### Example 14

The surgical instrument (10) of Example 13, wherein the controller is configured to detect the tissue characteristic at least by measuring a change in impedance as the end effector (12) clamps down to grasp the tissue.

### Example 15

The surgical instrument (10) of claim 14, wherein the controller is configured to:
provide an alternating current (AC) signal to a particular electrical pad among the plurality of electrical pads (904);
measure a voltage difference between the particular electrical pad and a reference electrical pad provided on the end effector (12); and
detect the change in impedance based on the voltage difference.

### Example 16

A surgical instrument (10), comprising:
a shaft (22);
an end effector (12) extending from the shaft (22), the end effector (12) comprising:
   a first jaw (16); and
   a second jaw (18), wherein at least one of the first jaw (16) and the second jaw (18) is movable relative to the other to grasp tissue;
   one or more electrical pads (904) positioned, on one or more tissue engaging surfaces of the end effector (12), externally adjacent to a target region (910) of the end effector (12); and
a controller (906) coupled to the one or more electrical pads (904) so as to receive signals from the one or more electrical pads (904), the controller (906) configured to:
   detect, based on the signals received from the one or more electrical pads (904), whether tissue is present outside of the target region (910) of the end effector (12); and
   in response to detecting that tissue is present outside of the target region (910), provide an alert signal to a user of the surgical instrument (10).

### Example 17

The surgical instrument (10) of Example 16, wherein the one or more electrical pads (904) positioned externally adjacent to the target region (910) comprise:
a first electrical pad positioned distally adjacent to the target region (910); and
a second electrical pad positioned proximally adjacent to the target region (910).

### Example 18

The surgical instrument (10) of Example 17, wherein:
the first electrical pad positioned distally adjacent to the target region (910) is configured to provide a signal indicative of whether the tissue that is present in the target region (910) extends beyond a distal end of the target region (910); and
the second electrical pad positioned proximally adjacent to the target region (910) is configured to provide a signal indicative of whether the tissue that is present in the target region (910) extends beyond a proximal end of the target region (910).

### Example 19

The surgical instrument (10) of Example 17 or 18, wherein the first electrical pad and the second electrical pad are electrically connected with one another to provide a single signal indicative of whether the tissue that is present in the target region (910) extends beyond a distal end or a proximal end of the target region (910).

### Example 20

A method for determining positioning of tissue grasped between a first jaw (16) and a second jaw (18) of an end effector (12) of a surgical instrument (10), the method comprising:
detecting (1302), by a controller (906), based on signals received from one or more target electrical pads, among a plurality of electrical pads (904) positioned on one or more tissue engaging surfaces of the end effector (12), whether tissue is present in a target region (910) of the end effector (12), wherein the one or more target electrical pads are positioned within the target region (910) of the end effector (12);
detecting (1304), by the controller (906), based on signals received from one or more external electrical pads, among the plurality of electrical pads (904), whether tissue is present externally adjacent to the target region (910) of the end effector (12), wherein the one or more external electrical pads are positioned externally adjacent to the target region (910) of the end effector (12); and
providing (1306), by the controller (906) based on whether tissue is present in the target region (910) and whether tissue is present externally adjacent to the target region (910), an indication of one or both of i) whether tissue is present in the target region (910) and ii) whether the tissue that is present in the target region (910) is contained entirely within the target region (910) to a user of the surgical instrument (10).

### Example 21

The method of Example 20, wherein detecting whether tissue is present in the target region (910) of the end effector (12) comprises detecting a tissue characteristic using the one or more target electrical pads as the end effector (12) clamps down to grasp the tissue.

### Example 22

The method of Example 21, wherein detecting the tissue characteristic comprises measuring a change in impedance as the end effector (12) clamps down to grasp the tissue.

### Example 23

The method of Example 22, wherein measuring a change in impedance as the end effector (12) clamps down to grasp the tissue comprises:
providing an alternating current (AC) signal to a particular electrical pad among the plurality of electrical pads (904);
measuring a voltage difference between the particular electrical pad and a reference electrical pad provided on the end effector (12); and
detecting the change in impedance based on the voltage difference.

The following clauses also relate to various non-exhaustive ways in which the teachings herein may be combined or applied.
1. A surgical instrument (10), comprising:
   a shaft (22);
   an end effector (12) extending from the shaft (22), the end effector (12) comprising:
      a first jaw (16); and
      a second jaw (18), wherein at least one of the first jaw (16) and the second jaw (18) is movable relative to the other to grasp tissue;
      a plurality of electrical pads (904) positioned, on one or more tissue engaging surfaces of the end effector (12), between a proximal end and a distal end of the end effector (12), the plurality of electrical pads (904) including i) one or more target electrical pads positioned within a target region (910) defined for tissue placement between the first jaw (16) and the second jaw (18) of the end effector (12) and ii) one or more external electrical pads positioned externally adjacent to the target region (910); and
   a controller (906) coupled to the plurality of electrical pads (904) so as to receive signals from the electrical pads (904), the controller (906) configured to:
      detect, based on the signals received from the electrical pads (904), tissue presence and positioning between the first jaw (16) and the second jaw (18) of the end effector (12), the controller (906) being configured to detect at least i) whether tissue is present in the target region (910) and ii) whether the tissue that is present in the target region (910) is contained entirely within the target region (910) and not present external to the target region (910); and
      provide an indication of the tissue presence and positioning to a user of the surgical instrument (10).
2. The surgical instrument (10) of claim 1, wherein the one or more target electrical pads positioned within the target region (910) comprise:
   a first target electrical pad extending longitudinally through a distal half of the target region (910), the first target electrical pad configured to provide a signal indicative of whether tissue is present in the distal half of the target region (910); and
   a second target electrical pad extending longitudinally through a proximal half of the target region (910), the second target electrical pad configured to provide a signal indicative of whether tissue is present in the proximal half of the target region (910).
3. The surgical instrument (10) of claim 1, wherein the one or more target electrical pads positioned within the target region (910) comprise a single target electrical pad extending longitudinally between a distal end of the target region (910) and a proximal end of the target region (910).
4. The surgical instrument (10) of claim 1, wherein the one or more external electrical pads positioned externally adjacent to the target region (910) comprise:
   a first external electrical pad positioned distally adjacent to the target region (910), the first external electrical pad configured to provide a signal indicative of whether the tissue that is present in the target region (910) extends beyond a distal end of the target region (910); and
   a second external electrical pad positioned proximally adjacent to the target region (910), the second external electrical pad configured to provide a signal indicative of whether the tissue that is present in the target region (910) extends beyond a proximal end of the target region (910).
5. The surgical instrument (10) of claim 4, wherein the first external electrical pad and the second external electrical pad are electrically connected with one another to provide a single signal indicative of whether the tissue that is present in the target region (910) extends beyond a distal end of the end effector (12) or is bunched up at a proximal end of the end effector (12).
6. The surgical instrument (10) of claim 1, wherein respective ones of the one or more target electrical pads are relatively greater in length as compared to respective ones of the one or more external electrical pads.
7. The surgical instrument (10) of claim 1, wherein the controller (906) is coupled to the plurality of electrical pads (904) using a number of connections less than a number of electrical pads among the plurality of electrical pads (904).
8. The surgical instrument (10) of claim 1, wherein the controller (906) is configured to detect the tissue presence and positioning between the first jaw (16) and the second jaw (18) of the end effector (12) based on measurements of tissue impedance in the target region (910) and externally adjacent to the target region (910).
9. The surgical instrument (10) of claim 1, further comprising a staple cartridge installed into a channel in one of the first jaw (16) and the second jaw (18) of the end effector (12), wherein the plurality of electrical pads (904) are disposed on one or both of the staple cartridge and the channel into which the staple cartridge is installed in the end effector (12).
10. The surgical instrument (10) of claim 1, further comprising:
   a handle portion (20) connected to the shaft (22), wherein the controller (906) is contained in the handle portion (20); and
   a plurality of conductors (908) that run through a cavity in the shaft (22) to couple the plurality of electrical pads (904) in the end effector (12) to the controller (906) contained in the handle portion (20).
11. The surgical instrument (10) of claim 10, wherein:
   the handle portion (20) is equipped with one or more indicators; and
   the controller (906) is configured to cause a tissue positioning indication to be provided to the user via the one or more indicators.
12. The surgical instrument (10) of claim 1, further comprising a transmitter coupled to the controller (906), wherein the transmitter is configured to wirelessly transmit tissue positioning signals generated using the plurality of electrical pads (904) to a processor of a computer, the processor being configured to one or both of i) further process the tissue positioning signals or ii) cause the indication of tissue positioning, determined based on the tissue positioning signals, to be displayed on a display coupled to the computer.
13. A surgical instrument (10), comprising:
   a shaft (22);
   an end effector (12) extending from the shaft (22), the end effector (12) comprising:
      a first jaw (16); and
      a second jaw (18), wherein at least one of the first jaw (16) and the second jaw (18) is movable relative to the other to grasp tissue;
      one or more electrical pads (904) positioned, on one or more tissue engaging surfaces of the end effector (12), externally adjacent to a target region (910) of the end effector (12); and
   a controller (906) coupled to the one or more electrical pads (904) so as to receive signals from the one or more electrical pads (904), the controller (906) configured to:
      detect, based on the signals received from the one or more electrical pads (904), whether tissue is present outside of the target region (910) of the end effector (12); and
      in response to detecting that tissue is present outside of the target region (910), provide an alert signal to a user of the surgical instrument (10).
14. The surgical instrument (10) of claim 13, wherein the one or more electrical pads (904) positioned externally adjacent to the target region (910) comprise:
   a first electrical pad positioned distally adjacent to the target region (910); and
   a second electrical pad positioned proximally adjacent to the target region (910).
15. The surgical instrument (10) of claim 14, wherein:
   the first electrical pad positioned distally adjacent to the target region (910) is configured to provide a signal indicative of whether the tissue that is present in the target region (910) extends beyond a distal end of the target region (910); and
   the second electrical pad positioned proximally adjacent to the target region (910) is configured to provide a signal indicative of whether the tissue that is present in the target region (910) extends beyond a proximal end of the target region (910).
16. The surgical instrument (10) of claim 14, wherein the first electrical pad and the second electrical pad are electrically connected with one another to provide a single signal indicative of whether the tissue that is present in the target region (910) extends beyond a distal end or a proximal end of the target region (910).
17. A method for determining positioning of tissue grasped between a first jaw (16) and a second jaw (18) of an end effector (12) of a surgical instrument (10), the method comprising:
   detecting (1302), by a controller (906), based on signals received from one or more target electrical pads, among a plurality of electrical pads (904) positioned on one or more tissue engaging surfaces of the end effector (12), whether tissue is present in a target region (910) of the end effector (12), wherein the one or more target electrical pads are positioned within the target region (910) of the end effector (12);
   detecting (1304), by the controller (906), based on signals received from one or more external electrical pads, among the plurality of electrical pads (904), whether tissue is present externally adjacent to the target region (910) of the end effector (12), wherein the one or more external electrical pads are positioned externally adjacent to the target region (910) of the end effector (12); and
   providing (1306), by the controller (906) based on whether tissue is present in the target region (910) and whether tissue is present externally adjacent to the target region (910), an indication of one or both of i) whether tissue is present in the target region (910) and ii) whether the tissue that is present in the target region (910) is contained entirely within the target region (910) to a user of the surgical instrument (10).
18. The method of claim 17, wherein detecting whether tissue is present in the target region (910) of the end effector (12) comprises detecting a tissue characteristic using the one or more target electrical pads as the end effector (12) clamps down to grasp the tissue.
19. The method of claim 18, wherein detecting the tissue characteristic comprises measuring a change in impedance as the end effector (12) clamps down to grasp the tissue.
20. The method of claim 19, wherein measuring a change in impedance as the end effector (12) clamps down to grasp the tissue comprises:
   providing an alternating current (AC) signal to a particular electrical pad among the plurality of electrical pads (904);
   measuring a voltage difference between the particular electrical pad and a reference electrical pad provided on the end effector (12); and
   detecting the change in impedance based on the voltage difference.

### IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Furthermore, any one or more of the teachings herein may be combined with any one or more of the teachings disclosed in U.S. Pat. App. No. 63/467,622, entitled “Surgical Stapler Cartridge Having Intermediate Raised Tissue Engagement Protrusions,” filed on May 19, 2023; U.S. Pat. App. No. 63/467,623, entitled “Surgical Stapler Cartridge Having Tissue Engagement Protrusions with Enlarged Engagement Surface,” filed on May 19, 2023; U.S. Pat. App. No. 63/467,648, entitled “Surgical Stapler Cartridge Having Raised Surface to Promote Buttress Adhesion,” filed on May 19, 2023; U.S. Pat. App. No. 63/467,469, entitled “Surgical Stapler Cartridge Having Cartridge Retention Features,” filed on May 19, 2023; U.S. Pat. App. No. 63/459,739, entitled “Surgical Stapler Anvil Having Staple Forming Pockets with Laterally Varying Orientations,” filed on May 19, 2023; U.S. Pat. App. No. 63/467,656, entitled “Surgical Stapler With Discretely Positionable Distal Tip,” filed on May 19, 2023; and/or U.S. Pat. App. No. 63/467,615, entitled “Incompatible Staple Cartridge Use Prevention Features for Surgical Stapler,” filed on May 19, 2023.

Additionally, any one or more of the teachings herein may be combined with any one or more of the teachings disclosed in U.S. Pat. App. No. 63/459,739, entitled “Surgical Stapler Anvil Having Staple Forming Pockets with Laterally Varying Orientations,” filed on Apr. 17, 2023. The disclosure of each of these U.S. patent applications is incorporated by reference herein in its entirety.

Additionally, any one or more of the teachings herein may be combined with any one or more of the teachings disclosed in U.S. Pat. No. 11,304,697, entitled “Surgical Stapler with Deflectable Distal Tip,” issued Apr. 19, 2022, the disclosure of which is incorporated by reference herein, in its entirety; U.S. Pat. No. 11,317,912, entitled “Surgical Stapler with Rotatable Distal Tip,” issued May 3, 2022, the disclosure of which is incorporated by reference herein, in its entirety; and/or U.S. Pat. No. 11,439,391, entitled “Surgical Stapler with Toggling Distal Tip,” issued Sep. 13, 2022, the disclosure of which is incorporated by reference herein, in its entirety.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as those made available by Auris Health, Inc. of Redwood City, CA or by Intuitive Surgical, Inc., of Sunnyvale, California.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1-15. (canceled)

16. A surgical instrument, comprising:
a shaft;
an end effector extending from the shaft, the end effector comprising:
a first jaw; and
a second jaw, wherein at least one of the first jaw and the second jaw is movable relative to the other to grasp tissue;
a plurality of electrical pads positioned, on one or more tissue engaging surfaces of the end effector, between a proximal end and a distal end of the end effector, the plurality of electrical pads including i) one or more target electrical pads positioned within a target region defined for tissue placement between the first jaw and the second jaw of the end effector and ii) one or more external electrical pads positioned externally adjacent to the target region, wherein the target region is a stapling region of the end effector and the one or more external electrical pads are positioned external to the stapling region of the end effector; and
a controller coupled to the plurality of electrical pads so as to receive signals from the electrical pads, the controller configured to:
detect, based on the signals received from the electrical pads, tissue presence and positioning between the first jaw and the second jaw of the end effector, the controller being configured to detect at least i) whether tissue is present in the target region and ii) whether the tissue that is present in the target region is contained entirely within the target region and not present external to the target region; and
provide an indication of the tissue presence and positioning to a user of the surgical instrument.

17. The surgical instrument of claim 16, wherein the one or more target electrical pads positioned within the target region comprise:
a first target electrical pad extending longitudinally through a distal half of the target region, the first target electrical pad configured to provide a signal indicative of whether tissue is present in the distal half of the target region; and
a second target electrical pad extending longitudinally through a proximal half of the target region, the second target electrical pad configured to provide a signal indicative of whether tissue is present in the proximal half of the target region.

18. The surgical instrument of claim 16, wherein the one or more target electrical pads positioned within the target region comprise a single target electrical pad extending longitudinally between a distal end of the target region and a proximal end of the target region.

19. The surgical instrument of claim 16, wherein the one or more external electrical pads positioned externally adjacent to the target region comprise:
a first external electrical pad positioned distally adjacent to the target region, the first external electrical pad configured to provide a signal indicative of whether the tissue that is present in the target region extends beyond a distal end of the target region; and
a second external electrical pad positioned proximally adjacent to the target region, the second external electrical pad configured to provide a signal indicative of whether the tissue that is present in the target region extends beyond a proximal end of the target region.

20. The surgical instrument of claim 19, wherein the first external electrical pad and the second external electrical pad are electrically connected with one another to provide a single signal indicative of whether the tissue that is present in the target region extends beyond the distal end of the target region of the end effector or is bunched up at a proximal end of the end effector.

21. The surgical instrument of claim 16, wherein respective ones of the one or more target electrical pads are relatively greater in length as compared to respective ones of the one or more external electrical pads.

22. The surgical instrument of claim 16, wherein the controller is coupled to the plurality of electrical pads using a number of connections less than a number of electrical pads among the plurality of electrical pads.

23. The surgical instrument of claim 16, wherein the controller is configured to detect the tissue presence and positioning between the first jaw and the second jaw of the end effector based on measurements of tissue impedance in the target region and externally adjacent to the target region.

24. The surgical instrument of claim 16, further comprising a staple cartridge installed into a channel in one of the first jaw and the second jaw of the end effector, wherein the plurality of electrical pads are disposed on one or both of the staple cartridge and the channel into which the staple cartridge is installed in the end effector.

25. The surgical instrument of claim 16, further comprising:
a handle portion connected to the shaft, wherein the controller is contained in the handle portion; and
a plurality of conductors that run through a cavity in the shaft to couple the plurality of electrical pads in the end effector to the controller contained in the handle portion.

26. The surgical instrument of claim 25, wherein:
the handle portion is equipped with one or more indicators; and
the controller is configured to cause a tissue positioning indication to be provided to the user via the one or more indicators.

27. The surgical instrument of claim 16, further comprising a transmitter coupled to the controller, wherein the transmitter is configured to wirelessly transmit tissue positioning signals generated using the plurality of electrical pads to a processor of a computer, the processor being configured to one or both of i) further process the tissue positioning signals or ii) cause the indication of tissue positioning, determined based on the tissue positioning signals, to be displayed on a display coupled to the computer.

28. A surgical instrument, comprising:
a shaft;
an end effector extending from the shaft, the end effector comprising:
a first jaw; and
a second jaw, wherein at least one of the first jaw and the second jaw is movable relative to the other to grasp tissue;
a plurality of electrical pads positioned, on one or more tissue engaging surfaces of the end effector, the plurality of electrical pads including i) one or more target electrical pads positioned within a target region defined for tissue placement between the first jaw and the second jaw of the end effector and ii) one or more external electrical pads positioned externally adjacent to the target region of the end effector, wherein the target region is a stapling region of the end effector and the one or more external electrical pads are positioned external to the stapling region of the end effector; and
a controller coupled to the plurality of electrical pads so as to receive signals from the plurality of electrical pads, the controller configured to:
detect, based on the signals received from the plurality of electrical pads, whether tissue that is present in the target region of the end effector extends outside of the target region of the end effector; and
in response to detecting that tissue extends outside of the target region, provide an alert signal to a user of the surgical instrument.

29. The surgical instrument of claim 28, wherein the plurality of electrical pads positioned externally adjacent to the target region comprise:
a first electrical pad positioned distally adjacent to the target region; and
a second electrical pad positioned proximally adjacent to the target region.

30. The surgical instrument of claim 29, wherein:
the first electrical pad positioned distally adjacent to the target region is configured to provide a signal indicative of whether the tissue that is present in the target region extends beyond a distal end of the target region; and
the second electrical pad positioned proximally adjacent to the target region is configured to provide a signal indicative of whether the tissue that is present in the target region extends beyond a proximal end of the target region.

31. The surgical instrument of claim 29, wherein the first electrical pad and the second electrical pad are electrically connected with one another to provide a single signal indicative of whether the tissue that is present in the target region extends beyond a distal end or a proximal end of the target region.

32. A method for determining positioning of tissue grasped between a first jaw and a second jaw of an end effector of a surgical instrument, the method comprising:
detecting, by a controller, based on signals received from one or more target electrical pads, among a plurality of electrical pads positioned on one or more tissue engaging surfaces of the end effector, whether tissue is present in a target region of the end effector, wherein the one or more target electrical pads are positioned within the target region of the end effector;
detecting, by the controller, based on signals received from one or more external electrical pads, among the plurality of electrical pads, whether tissue is present externally adjacent to the target region of the end effector, wherein the target region is a stapling region of the end effector and the one or more external electrical pads are positioned external to the stapling region of the end effector; and
providing, by the controller based on whether tissue is present in the target region and whether tissue is present externally adjacent to the target region, an indication of one or both of i) whether tissue is present in the target region and ii) whether the tissue that is present in the target region is contained entirely within the target region to a user of the surgical instrument.

33. The method of claim 32, wherein detecting whether tissue is present in the target region of the end effector comprises detecting a tissue characteristic using the one or more target electrical pads as the end effector clamps down to grasp the tissue.

34. The method of claim 33, wherein detecting the tissue characteristic comprises measuring a change in impedance as the end effector clamps down to grasp the tissue.

35. The method of claim 34, wherein measuring a change in impedance as the end effector clamps down on the tissue comprises:
providing an alternating current (AC) signal to a particular electrical pad among the plurality of electrical pads;
measuring a voltage difference between the particular electrical pad and a reference electrical pad provided on the end effector; and
detecting the change in impedance based on the voltage difference.
